(19) United States
(12) Patent Application Publication
(10) Pub. No.: US 2004/0082068 A1
(43) Pub. Date: Apr. 29, 2004

(54) Incorporation and priming function of trnalys in hiv and related viruses

(76) Inventors: Lawrence Kleiman, Cote St-Luc (CA); Shan Chen, Montreal (CA)

Correspondence Address: Goudreau Gage Dubuc, Stock Exchange Tower, Suite 3400, P O Box 242 800 Place-Victoria, Montreal, QC, H4Z 1E9, CA

(21) Appl. No.: 10/432,223

(22) PCT Filed: Nov. 27, 2001
(86) PCT No.: PCT/CA01/01700

(51) Int. Cl. C12N 15/867; C12N 9/10

(52) U.S. Cl. 435/456; 435/193

(57) ABSTRACT

The present invention relates to the demonstration of a direct relationship between the amount of tRNA^{Lys3} packaged into HIV, the amount of tRNA^{Lys3} placed onto the reverse transcriptase primer binding site which can initiate reverse transcription, and viral infectivity. The present invention also relates to the incorporation of lysyl tRNA synthase into HIV-1 and to the aminoacylation status of tRNA^{Lys3} and its impact on virion incorporation. The present invention also relates to methods of modulating lysyl tRNA synthetase (LysRS)-facilitated processes associated with tRNA^{Lys3} priming function of RT, to bioassays to screen and identify compounds which interfere with these processes and to compositions for modulating these processes. In one particular embodiment, the compositions modulate the incorporation of LysRS and/or tRNA^{Lys3} into HIV and related virions. The present invention also relates to aaRS-facilitated processes associated with their cognate tRNA^{aa} priming function in other types of retroviruses and to methods, assays and compositions which modulate them.

### FIELD OF THE INVENTION

The present invention relates to the incorporation and priming function of lysyl tRNA^{Lys} in HIV-1 and related viruses. The present invention also relates to methods of modulating the incorporation of tRNA^{Lys} and/or priming function into HIV-1, to bioassays to screen and identify compounds which interfere with the incorporation of tRNA^{Lys3} and/or priming function thereof into HIV and related virions, to methods of screening and identification of such compounds and to compositions for modulating the incorporation and/or priming function thereof of tRNA^{Lys} into HIV and related virions.

### BACKGROUND OF THE INVENTION

During HIV-1 assembly, viral particles are formed at the membrane by the precursor protein Pr55^{gag}. This protein is then processed during viral maturation into matrix (MAp17), capsid (CAp24), nucleocapsid (NCp7), and p6 (Swanstrom et al., 1997). Another precursor protein, Pr160^{gag-pol}, is also assembled into the Gag particle, and its proteolytic processing gives rise to MAp17, CAp24, NCp7, and the enzymes of HIV-1, protease (PRp11), reverse transcriptase (RTp66/p51), and integrase (INp11) (Swanstrom et al., 1997). Also incorporated into the viral particle are genomic RNA and cellular tRNA^{Lys}. Both major tRNA^{Lys} isoacceptors, tRNA^{Lys3} and tRNA^{Lys1,2}, are selectively packaged into the virus (Jiang et al., 1993). While the function of tRNA^{Lys1,2} in the viral life cycle remains unknown, tRNA^{Lys3} is used as the primer for the reverse transcriptase-catalyzed synthesis of minus strand DNA (Leis et al., 1993). Placement of the primer tRNA^{Lys3} onto the primer binding site (PBS) on the viral genome, and infectivity of the viral population, are both directly proportional to the amount of viral tRNA^{Lys3} packaged into the viruses. Thus, tRNA^{Lys} and more particularly tRNA^{Lys3} play a pivotal role in the infectivity of HIV.

The selective packaging of tRNA^{Lys} into HIV-1 occurs independently of genomic RNA packaging (Jiang et al., 1993) or precursor protein processing (Mak et al., 1994), but does depend on the presence of Pr160^{gag-pol} (Mak et al., 1994). Since reverse transcriptase (RT) is known to bind to primer tRNA^{Lys3}, RT sequences within Pr160^{gag-pol} are candidate for binding tRNA^{Lys3}. More specifically, cross-linking studies indicate that sequences within the thumb subdomain of RT appear to play a role in the in vitro binding of purified tRNA^{Lys3} to purified RTp66,p51 (Dufour et al., 1999), while in vivo studies indicate a role for the thumb subdomain sequences in Pr160^{gag-pol} in binding to tRNA^{Lys3} during packaging (Khorchid et al., 2000). In this in vivo study, a C-terminal deletion of Pr160^{gag-pol}, which removes the integrase domain, and the RNaseH and connection subdomains of RT, does not affect tRNA^{Lys} packaging, but additional removal of the thumb subdomain sequences in RT abolishes selective tRNA^{Lys} packaging.

NCp7 sequences within Pr55^{gag} or Pr160^{gag-pol} are other candidates for binding to tRNA^{Lys} during packaging. However, NCp7 mutations, specifically in Pr160^{gag-pol}, do not appear to affect tRNA^{Lys} packaging (Huang et al., 1994), while NCp7 mutations in Pr55^{gag} which disrupt tRNA^{Lys} packaging do so by disrupting Gag particle formation (Huang et al., 1994). Pr55^{gag} is required to form viral particles, and binds to Pr160^{gag-pol}, but separating these functions from specific Pr55^{gag} sequences whose function is to bind to tRNA^{Lys} has not yet been possible. Evidence for an interaction between Pr55^{gag} and tRNA^{Lys3} has not been provided from tRNA^{Lys3} packaging studies, but from tRNA^{Lys3} placement studies which indicate that this protein, and not Pr160^{gag-pol}, plays a major role in placement of tRNA^{Lys3} onto the PBS in vitro (Feng et al., 1999) or in vivo (Cen et al., 1999).

In considering the interactions involved between viral proteins and tRNA^{Lys} during packaging, the fact that tRNAs, like other RNAs, exist in the cytoplasm bound to proteins must be taken into account. For tRNAs, a major protein binding partner in the cytoplasm is its cognate amino acyl tRNA synthetase. LysRS has been shown to exist as truncated forms in eukaryotes.

In any event, there remains a need to elucidate how tRNA^{Lys} is packaged into HIV virions (or how other tRNAs involved in RT priming are packaged in other retroviruses). Because of the important role of tRNA^{Lys} in the life cycle of HIV, and more particularly in RT priming, the identification of the factor responsible for the packaging of tRNA^{Lys} could open the way to retroviral infectivity modulation, HIV infectivity modulation, transport of molecules into retroviruses and anti-retroviral therapy.

More particularly, there remains a need to elucidate how tRNA^{Lys3} functions as a primer for reverse transcriptase, how it is selectively packaged into HIV-1, annealed to the primer binding site, and used to initiate reverse transcription.

There also remains a need to modulate the incorporation of tRNA^{Lys} into HIV virions.

The present invention seeks to meet these and other needs.

The present description refers to a number of documents, the content of which is herein incorporated by reference in their entirety.

### SUMMARY OF THE INVENTION

The invention concerns the following: 1) Correlated with the selective packaging of tRNA^{Lys} into the virus during viral assembly, the major tRNA^{Lys}-binding protein, LysRS is also non-randomly packaged into HIV-1; 2) The amount of tRNA^{Lys} incorporated into the virion is limited by the amount of LysRS packaged; 3) Annealing of tRNA^{Lys3} to the primer binding site (PBS), and resulting viral infectivity are directly proportional to the amount of tRNA^{Lys3} in the virus; 4) During or after incorporation into the virus, LysRS is cleaved by a non-viral protease; and 5) the incorporation of tRNA^{Lys3} into HIV-1 positively correlates with its aminoacylation status.

Thus, broadly, the present invention relates to assays and methods making use of the fact that the infectivity of a retrovirus and more specifically RT function can be modulated by a modulation of the incorporation thereinto of the tRNA involved in reverse transcriptase priming, through its cognate aminoacyl tRNA synthetase.

The invention also relates to the fact that in HIV transfected COS cells and in HIV chronically infected cell lines there is a variation in the amount of tRNA^{Lys} in the virion, and the fact that there is a positive correlation between the level of tRNA^{Lys} in the virion, the placement of tRNA^{Lys} onto the RT primer binding site or other RNA genomic region and the infectivity of HIV. Further, an artificial variation of tRNA^{Lys3} in the virus by cotransfection of COS cells with genes coding for tRNA^{Lys3} is shown to increase viral tRNA^{Lys3}, tRNA^{Lys3} placement, and viral infectivity. Alternatively, cotransfection of the same cells with genes coding for tRNA^{Lys2} which acts as a “dominant negative” is shown to lower viral tRNA^{Lys3} incorporation, tRNA^{Lys3} placement and viral infectivity. Of note, increasing LysRS packaging in the virion results in an increase in all tRNA^{Lys} isoacceptors packaging, indicating that LysRS plays a limiting role in tRNA^{Lys} packaging, and also results in increased tRNA^{Lys3} placement and viral infectivity. In addition, there is also a positive correlation between the aminoacylation status of tRNA^{Lys3} and HIV-1 infectivity.

The present invention further relates to the fact that the form of LysRS associated with the particles (or virions) virus is smaller than that found in the cytoplasm. While the full length (large species) is the major form found in the cytoplasm, the intermediate size form is the major species packaged in the virus. Epitope tagging indicates that the N-terminal region of LysRS has been lost in the intermediate species.

LysRS is matured (e.g. processed) into three different size species. Since the intermediate form is preferably associated with the virion, as compared to the larger form thereof predominantly found in the cytoplasm, the invention in addition relates to means of modulating the incorporation or packaging of tRNA^{Lys} into HIV virions by modulating the interaction between tRNA^{Lys} and LysRS. In addition, the present invention relates to means of modulating tRNA^{Lys3} priming function not only by modulating incorporation of tRNA^{Lys}/LysRS into virions, but also by altering or inhibiting the processing of LysRS into a smaller form. In a particular embodiment, the invention relates to a method of modulating incorporation of tRNA^{Lys} into HIV virions by modulating the processing of LysRS into the intermediate form.

The present invention further relates to means to target molecules to mature HIV virions and more particularly into HIV-1 and/or HIV-2 virions to affect their structural organization and/or functional integrity.

In addition, the present invention relates to a LysRS protein or fragments thereof which enable the development of chimeric molecules that can be specifically targeted into mature HIV virions and more particularly into HIV-1 and/or HIV-2 virions and other lentiviruses to affect their structural organization and/or functional integrity, thereby resulting in treatment of HIV and related viruses and more particularly of HIV-1 and/or lentiviruses infections.

In a particular embodiment of the present invention, there is provided a method of modulating the infectivity of a retrovirus by modulating aminoacyl tRNA synthetase-facilitated processes associated with its cognate tRNA priming function, including at least one of: a) cognate tRNA incorporation into the virion, b) annealing thereof to the PBS or other viral RNA regions; and c) priming of RT. In a particularly preferred embodiment of the present invention, the retrovirus is a lentivirus and even more particularly, HIV-1. In a particular embodiment, the infectivity of HIV-1 is affected by modulating tRNA^{Lys3} function in the HIV-1 virion by modulating its incorporation thereinto by LysRS.

Further, the invention relates to a therapeutic agent which permits the targeting of chimeric molecules into HIV-1 and/or HIV-2 virions as a treatment for HIV-1 and/or HIV-2 infections.

Also, the present invention relates to the identification of RNA-protein and/or protein-protein interactions responsible for tRNA^{Lys3} priming function and/or incorporation into mature HIV-1 and/or other lentiviruses.

Yet another aspect of the present invention relates to means to incorporate tRNA^{Lys} and/or LysRS or chimeras thereof into the mature HIV-1 and/or HIV-2 virions by making use of the interactions responsible for incorporation of tRNA^{Lys} or LysRS therein, thereby affecting the functional integrity of the HIV virions.

In an additional aspect, the present invention relates to a LysRS protein fragment, and/or a tRNA^{Lys} RNA fragment, which permit the development of molecules that can specifically interfere with the interactions responsible for tRNA^{Lys} priming function and/or incorporation into HIV virions or related virions and more particularly into HIV-1 and/or other lentiviruses, to affect their functional integrity (e.g. resulting in treatment of HIV, HIV-related viruses, HIV-1 and/or lentiviral infections).

In addition, the invention relates to a therapeutic agent which interferes with the processes associated with tRNA^{Lys3} priming function which are facilitated by LysRS. In one embodiment, the invention relates to a therapeutic agent which interferes with the interactions responsible between a tRNA involved in RT priming/incorporation in a retrovirus, and its cognate amino acyl tRNA, as a retroviral treatment and more particularly which interferes with the interaction between tRNA^{Lys3} and LysRS, thereby decreasing or inhibiting incorporation of tRNA^{Lys3} into HIV (or related viruses) virions as a treatment for HIV or related viral infections.

Also, the present invention relates to an assay which enables the screening and identification of molecules which modulate the LysRS-facilitated processes associated with tRNA^{Lys3} priming function. The interaction which is targeted in these assays is selected from the group consisting of: LysRS and tRNA^{Lys}; LysRS and Gag, LysRS and the non-viral protease responsible for its processing. In a particular embodiment, the invention provides screening assays to identify agents which interfere with the interaction between an aminoacyl tRNA synthetase and its cognate tRNA involved in RT priming. In one particular embodiment, the invention relates to a simple, rapid and high-throughput assay for the screening and identification of molecules which modulate the interaction between an aminoacyl tRNA and its cognate tRNA involved in RT priming and more particularly between LysRS and tRNA^{Lys}.

Before the present invention, it was not known that LysRS was incorporated into HIV virions. Also, prior to the teachings of the present invention, the molecule involved in the packaging of tRNA^{Lys} into HIV virion was unknown.

In accordance with one embodiment of the present invention, there is therefore provided a method of modulating an aminoacyl tRNA synthetase-facilitated process associated with its cognate tRNA priming function wherein this process is selected from the group consisting of a) cognate tRNA incorporation into the retrovirus virion; b) annealing thereof to the primer binding site (PBS) or other retroviral RNA regions; and c) initiation of RT, comprising a modulation of the activity and/or of the level of a cognate aminoacyl tRNA synthetase, a modulation of cognate tRNA-aminoacyl tRNA synthetase interaction, a modulation of aminoacyl tRNA-Gag interaction, or a modulation of aminoacylation of the cognate tRNA, wherein the level and/or activity of the cognate aminoacyl tRNA synthetase, or aminoacylation level of the cognate tRNA in a cell infected by the retrovirus positively correlates with an incorporation of the tRNA into the virion and with the placement of the tRNA onto the retroviral genome and with infectivity of the retrovirus.

In accordance with another embodiment of the present invention, there is also provided, a method of targeting a molecule into HIV or other lentiviral virions comprising providing the molecule linked to a sufficient number of amino acids of LysRS in a cell infected with HIV or other lentiviruses, whereby incorporation of LysRS in the virions enables incorporation of the molecule thereinto.

In accordance with yet another embodiment of the present invention, there is provided a chimeric protein capable of being incorporated into HIV or other lentiviral virions, comprising a first and second portion, wherein the first portion comprises a sufficient number of amino acids of LysRS to enable incorporation of the chimeric protein into the virions.

In addition, in accordance with another embodiment of the present invention there is provided, a protein for interfering with an aminoacyl tRNA synthetase-facilitated process associated with its cognate tRNA priming function wherein this process is selected from the group consisting of: a) cognate tRNA incorporation into the retrovirus virion; b) annealing thereof to the PBS or other retroviral RNA regions; and c) initiation of RT, wherein the protein is expressed in trans with respect to the retroviral genome and comprises one of: a) an aminoacyl tRNA synthetase incorporation domain; b) the cognate tRNA molecule thereof, and c) a Gag precursor protein of the retroviral virion; and wherein the protein interferes with the incorporation of the native tRNA and/or native aminoacyl/tRNA synthetase into the virion, thereby reducing the infectivity of the retroviral virion.

Also, in accordance with another embodiment of the present invention there is provided, a method of screening and selecting an agent that modulates the incorporation of a tRNA and/or a cognate aminoacyl tRNA synthetase thereof into a retroviral virion comprising: a) incubating a candidate agent with a cell expressing at least a portion of the aminoacyl tRNA synthetase, the portion being sufficient for enabling incorporation into the virion; wherein the cell also contains the retroviral virion, such that the aminoacyl tRNA synthetase is capable of being incorporated into the virions; and b) determining the amount of the aminoacyl tRNA synthetase incorporated into the virons; wherein an agent that modulates the incorporation of the aminoacyl tRNA synthetase and/or tRNA into the virion is selected when the amount of incorporated aminoacyl tRNA synthetase in the presence of the candidate agent is measurably different than in the absence thereof.

In accordance with yet another embodiment of the present invention there is provided, a method for reducing the infectivity of a retrovirus, comprising a reduction in the incorporation of the tRNA involved in RT priming and/or of the cognate aminoacyl tRNA synthetase thereof. In accordance with a preferred embodiment of the present invention, there is provided a method for reducing the infectivity of HIV, comprising a reduction in the the LysRS-facilitated processes associated with tRNA^{Lys3} priming function.

The Applicant is the first to provide a formal demonstration that there is a positive correlation between the level of incorporation of LysRS, the level of incorporation of its cognate tRNA species involved in RT priming (tRNA^{Lys3}), and the aminoacylation level of tRNA^{Lys3} in HIV and the infectivity thereof, and that LysRS serves as a target used by HIV-1 proteins to selectively incorporate tRNA^{Lys} into the virions.

While the interaction of the instant invention is exemplified with HIV-1, it will be clear to the person of ordinary skill to which this invention pertains that in view of the conservation of the different HIV strains and other lentiviruses such as SIV, that the present invention has broader scope than to HIV-1. Thus, the terminology HIV should be interpreted as broadly refering to the large family of lentiviruses (e.g. HIV, SIV . . . ). In fact, in view of the fact that all lentiviruses use either tRNA^{Lys1}, 2 or tRNA^{Lys3} as the primer tRNA for reverse transcription, the present invention finds applications for all lentiviruses.

Since other retroviruses use other primer tRNAs (e.g. avian retroviruses use tRNA^{Trp} and murine leukemia viruses use tRNA^{Pro}), the present invention can be generalized to the use of the interaction between the specific tRNAs used to prime reverse transcription (RT) in retroviruses in general and their cognate aminoacyl tRNA synthetase, to modulate the infectivity, target molecules into virions and the like in retroviruses in general. In fact, the applicant has indeed shown that in the avian retrovirus, Rous Sarcoma Virus (RSV), the selective packaging of the tRNA used for priming reverse transcriptase, tRNA^{Trp}, is accompanied by a selective packaging of its cognate aminoacyl tRNA synthetase, tryptophanyl tRNA synthetase (TrpRS). Of note, the viral-associated form of TrpRS is smaller than that found in the cytoplasm.

Thus, the assays, methods and compositions of the present invention should not be limited to HIV.

While the present invention is demonstrated with tRNA^{Lys3}, the present invention should not be so limited. In fact, tRNA^{Lys1}, tRNA^{Lys2} and tRNA^{Lys3} are all selectively incorporated into HIV. TRNA^{Lys1} and tRNA^{Lys2} are often referred to as tRNA^{Lys1,2} since they differ by only one base pair in the anticodon stem. While very similar to tRNA^{Lys1,2}, tRNA^{Lys3} differs from these other two by 14 and 16 bases, respectively.

LysRS and other specific aminoacyl tRNA synthetase show a very significant conservation throughout evolution. Shiba et al., 1997, for example, demonstrate this conservation in FIG. 1 which shows an alignment of 21 evolutionary distinct LysRSs (bacteria, plants, animals). In fact, Shiba et al. 1997 even showed functional complementation of the aminoacylation activity of E. coli tRNA^{Lys} by transfecting thereinto human LysRS, demonstrating the evolutionary pressure on the maintenance of the structure function relationship of LysRS and tRNA^{Lys} and more broadly on aatRNA synthetases and their cognate tRNAs.

In view of the conservation of tRNA^{Lys3} and its cognate LysRS throughout evolution, the present invention should not be so limited to the use of human sequences thereof for the assays and methods of the present invention. As recited above, the present invention has a broad implication to retroviruses in general, for which priming of RT is dependent on a specific tRNA.

In order to provide a clear and consistent understanding of terms used in the present description, a number of definitions are provided hereinbelow.

The terminology “aminoacyl tRNA synthetase (e.g. LysRS)-facilitated processes associated with its cognate tRNA (e.g. tRNA^{Lys3})” is used herein to cover: a) incorporation of the tRNA into a retroviral virion; b) its annealing to the retroviral genome; c) its initiation of reverse transcription.

The terminology “non-viral protease” when referring to a protease which is responsible for processing LysRS (or other aaRSs) relates to a cellular protease which can be associated with the assembling virion or packaged into the virion so as to process the aaRS in the assembling virion or within the virion per se.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

The term “cognate” is used herein to refer to the specific recognition between a given aminoacyl tRNA synthetase and tRNA(s). One non-limiting example thereof is LysRS and its cognate tRNA^{Lys1,2} or tRNA^{Lys3}. The term “cognate” is similarly used to refer to a particular tRNA and its cognate aminoacyl tRNA synthetase (e.g. tRNA^{Lys3} and LysRS).

The term “LysRS incorporation domain” (or “aminoacyl tRNA synthetase incorporation domain”) refers herein to a sufficient portion of the amino acid sequence of the synthetase to enable its incorporation into a retrovirus.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA or RNA) for practicing the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised of the deoxyribonucleotides adenine (A), guanine (G), thymine (T) and/or cytosine (C), often in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction. Of course, as known in the art, numerous applications use single stranded nucleic acids.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence defining a single protein or polypeptide. A “structural gene”-defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation.

As used herein, the designation “functional derivative” denotes, in the context of a functional derivative of a sequence whether a nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. All these methods are well known in the art.

Of course, it will be recognized that in certain embodiments, the biological activity of LysRS, for example, to enable an incorporation of tRNA^{Lys3} into HIV virions could be destroyed while maintaining the interaction with tRNA^{Lys3}. Such a variant could be used as a dominant negative which titrates out the tRNA^{Lys3}. Of note, tRNA^{Lys1} and/or tRNA^{Lys2} overexpression has been shown to lower incorporation ot tRNA^{Lys3} (Example 1). Thus, such an overexpression could be used to lower the infectivity of HIV virions (also a type of dominant negative approach). This type of overexpression could be applied to retroviruses in general. Of course, the same applies to other tRNAs involved in RT priming in other retroviruses.

As used herein, “chemical derivatives” is meant to cover additional chemical moieties not normally part of the subject matter of the invention. Such moieties could affect the physico-chemical characteristic of the derivative (e.g. solubility, absorption, half life, decrease of toxicity and the like). Such moieties are exemplified in Remington's Pharmaceutical Sciences (1980). Methods of coupling these chemical-physical moieties to a polypeptide or nucleic acid sequence are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions associated with HIV injection.

Of course, the molecules can be in pools or in libraries and can be used in primary, secondary or tertiary screens. In one embodiment, the screening assays are automated.

As used herein, agonists and antagonists of LysRS-tRNA^{Lys3} interaction (or more broadly of aminoacyl tRNA synthetase-tRNA [involved in RT-priming] interaction, and/or interactions between gag precursor proteins and tRNA synthetase and/or tRNA) also include potentiators of known compounds with tRNA-tRNA-synthetase agonist or antagonist properties. In one embodiment, agonists can be detected by contacting the indicator cell with a compound or mixture or library of molecules for a fixed period of time is then determined.

The level of gene expression of the reporter gene (e.g. the level of luciferase, or β-gal, produced) within the treated cells can be compared to that of the reporter gene in the absence of the molecules(s). The difference between the levels of gene expression indicates whether the molecule(s) of interest agonizes the aforementioned interaction. The magnitude of the level of reporter gene product expressed (treated vs. untreated cells) provides a relative indication of the strength of that molecule(s) as an agonist. The same type of approach can also be used in the presence of an antagonist(s).

Alternatively, an indicator cell in accordance with the present invention can be used to identify antagonists. For example, the test molecule or molecules are incubated with the host cell in conjunction with one or more agonists held at a fixed concentration. An indication and relative strength of the antagonistic properties of the molecule(s) can be provided by comparing the level of gene expression in the indicator cell in the presence of the agonist, in the absence of test molecules v. in the presence thereof. Of course, the antagonistic effect of a molecule can also be determined in the absence of agonist, simply by comparing the level of expression of the reporter gene product in the presence and absence of the test molecule(s).

It shall be understood that the “in vivo”experimental model can also be used to carry out an “in vitro”assay. For example, cellular extracts from the indicator cells can be prepared and used in one of the aforementioned “in vitro” tests or others.

As used herein the recitation “indicator cells” refers to cells that express an aminoacyl tRNA synthetase and its cognate tRNA and in a preferred embodiment, the cognate tRNA involved in RT priming. In an especially preferred embodiment, the indicator cells express LysRS and tRNA^{Lys3}, and wherein an interaction between these domains is coupled to an identifiable or selectable phenotype or characteristic such that it provides an assessment of the interaction between the domains. Such indicator cells can be used in the screening assays of the present invention. In certain embodiments, the indicator cells have been engineered so as to express a chosen derivative, fragment, homolog, or mutant of these two interacting domains. The cells can be yeast cells or higher eukaryotic cells such as mammalian cells (WO 96/41169). In one embodiment, the indicator cells are yeast cells. In one particular embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of the two interacting domains. Such an indicator cell could be used to rapidly screen at high-throughput a vast array of test molecules. In a particular embodiment, the reporter gene is luciferase or β-Gal.

Of course, at least one of the interacting domains and in particular the virion incorporation domain of the present invention may be provided as a fusion protein. The design of constructs therefor and the expression and production of fusion proteins are well known in the art (Sambrook et al., 1989, supra; and Ausubel et al., 1994, supra. In a particularly preferred embodiment, the fusions are a LexA-LysRS fusion (DNA-binding domain-LysRS; bait) and a B42-tRNA^{Lys3} fusion (transactivator domain-tRNA^{Lys3}; prey). In still a particularly preferred embodiment, the LexA-LysRS and B42-tRNA^{Lys3} fusion proteins are expressed in a yeast cell also harboring a reporter gene operably linked to a LexA operator and/or LexA responsive element.

Non-limiting examples of such fusion proteins include hemaglutinin fusions and Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art.

As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of LysRS-tRNA and/or more particularly LysRS-tRNA^{Lys3} interaction.

In another embodiment, the virion incorporation domain of the present invention can be fused to an antiviral agent (a small molecule, chemical, macromolecule, etc.) or be part of a chimeric protein which also encodes an antiviral agent. In one embodiment, the protein comprising the LysRS incorporation region of the present invention further comprises a protein fragment covalently attached to its N- or C-terminal to form a chimeric protein which is also incorporated by the mature virion. Such an attached protein fragment of the present invention consists of amino acid sequence effective in reducing retroviral (e.g. HIV) expression or replication, the amino acid sequence encoding for example a RNase activity, protease activity, a sequence creating steric hindrance during virion assembly and morphogenesis and/or affecting viral protein interactions responsible for infectivity and/or viral replication.

In another embodiment, the protein of the present invention which targets same to the virion further comprises a molecule to form a protein-molecule complex which is also incorporated by the mature virion. Such a molecule is selected from the group consisting of anti-viral agents, RNases, proteases, and amino acid sequences capable of creating steric hindrance during virion assembly and morphogenesis. The molecule of the protein-molecule complex of the present invention affects the structural organization or functional integrity of the mature virion by steric hindrance or enzymatic disturbance of the virion.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, such an advantage might be rendered moot if both polypeptide tested directly interact. It will be understood that extracts from mammalian cells for example could be used in certain embodiments, to compensate for the lack of certain factors.

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins or chimeras thereof according to the present invention can be introduced into individuals in a number of ways. For example, erythropoietic cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

For administration to humans, the prescribing medical professional will ultimately determine the appropriate form and dosage for a given patient, and this can be expected to vary according to the chosen therapeutic regimen (e.g. DNA construct, protein, cells), the response and condition of the patient as well as the severity of the disease.

Composition within the scope of the present invention should contain the active agent (e.g. fusion protein, nucleic acid, and molecule) in an amount effective to achieve the desired therapeutic effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th-Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

In one embodiment, the present invention provides a simple, rapid high-throughput functional bioassay for identifying molecules that modulate the LysRS-tRNA^{Lys} interaction. These molecules can act either as agonists or antagonists of LysRS-tRNA^{Lys} interaction and incorporation of LysRS and/or tRNA^{Lys} inside HIV virions. In one embodiment, the assay is an “in vivo” experimental model based on the incubation of indicator cells with test molecules and the identification of the test molecule as agonist or antagonist of LysRS-tRNA Ys direct interaction. Alternatively, it is based on the use of an “in vitro” experimental model such as an enzymatic assay, binding assay and the like. Such assays are common and known to the person of ordinary skill. Molecules (or compounds) can be tested individually or in pools or libraries.

The term “antagonist” refers to a molecule which inhibits the interaction between LysRS-tRNA^{Lys}, thereby interfering with the incorporation of LysRS and/or tRNA^{Lys} into HIV virions.

Alternatively, the term “agonist” refers to a compound that stimulates such an incorporation by promoting LysRS-tRNA^{Lys} interaction.

The term “modulator” is used herein to refer to a molecule or a mixture or pool thereof which positively or negatively affects the direct LysRS-tRNA^{Lys} interaction.

In another embodiment, the rapid high throughput functional assay is used to screen and identify agonists or antagonists of LysRS-tRNA^{Lys}-Gag interactions and incorporation of LysRS and/or tRNA^{Lys} inside HIV virions or agonists or antagonists of LysRS processing into a smaller form. In such assays, “antagonist” refers to a molecule which inhibits the interactions between LysRS-tRNA^{Lys} and Gag, or LysRS processing. The terms “agonist” and “modulator” are used similarly in this context as when referring to the LysRS-tRNA^{Lys} interaction.

A preferred molecule used in accordance with the present invention may be selected from the group consisting of an anti-viral agent and/or a second amino acid sequence which contains a sufficient number of amino acids corresponding to RNases, proteases, or any protein capable of creating steric hindrance during virion morphogenesis and/or affecting viral protein interactions responsible for infectivity and/or viral replication.

In one embodiment, a chimeric protein comprising the LysRS domain enabling incorporation into HIV may be used for the targeting of molecules into the mature virions of HIV and more particularly into HIV-1 and/or HIV-2. Non-limiting examples of such molecules include polypeptides, proteins (e.g. proteases, nucleases), ribozymes, and anti-viral agents.

It should be understood by the person of ordinary skill that, in particular with the LysRS-tRNA^{Lys} interaction taught and exemplified herein, the invention should not be limited to human LysRS. Indeed, LysRS (and aminoacyl tRNA synthetases in general) are significantly conserved throughout evolution. For example, regarding conservation of LysRS, in a comparison of the sequence from 5 eukaryotic LysRS, shows that the catalytic region (the region that aminoacylates the tRNA) is very conserved. In addition, there is also a 60 aa N-terminus in eukaryotic LysRS which is not required for aminoacylation. Examples of sequence alignments can be found in Shiba et al., 1997 (J. Biol. Chem. 272:22809-22816). Thus, an assay could be based on the interaction between the catalytic region of aminoacyl tRNA synthetase and its cognate tRNA. In addition, cross-species complementation of the aminoacylation of tRNAs has been demonstrated, supporting the contention that the present invention has broad applicability and, for example, should not be limited to human LysRNA.

It is therefore an object of this invention to provide screening assays using LysRS (or another aminoacyl tRNA synthetase whose substrate is involved in RT priming) which can identify compounds which have a therapeutic benefit in reducing the infectivity of a retrovirus and especially of HIV and related viruses. This invention also claims those compounds, the use of these compounds in reducing infectivity of a retrovirus, and any use of any compounds identified using such a screening assay in reducing infectivity of a retrovirus.

Generally, high throughput screens for one or more aaRS i.e. candidate or test compounds or agents (e.g., peptides, peptidomimetics, small molecules or other drugs) may be based on assays which measure biological activity of aaRS, which measure its interaction with its cognate tRNA, which measures aaRS incorporation in a retroviral virion or which measures the level of processing of the aaRS into the form which is found in the virion. In addition, assays can also be set up to identify agents/molecules which modulate the incorporation of tRNA/aaRS incorporation (e.g. tRNA^{Lys}/LysRS in HIV) in a retrovirus by assaying the interaction between one of aaRS and/or tRNA and precursor proteins of the retrovirus. In a particular embodiment, such assays assess the interaction between one of LysRS and/or tRNA^{Lys} and Pr55^{gag} and/or Pr160^{gag-pol}. The invention therefore provides a method (also referred to herein as a “screening assay”) for identifying modulators, which have a stimulatory or inhibitory effect on, for example, aaRS biological activity or expression, or which bind to or interact with aaRS protein (or its cognate tRNA), or which have a stimulatory or inhibitory effect on, for example, the expression or activity of an enzyme involved in the processing of aaRS. As described above, screening assays can also identify molecules which modulate aaRS processing in a retrovirus by assessing the size of the aaRS in the presence or absence of the molecule.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261:1303; Carrell et al. (1994) Angew. Chem, Int. Ed Engl. 33:2059; Carell et al. (1994) Angew. Chem. Jnl. Ed. Engl. 33:2061; and in Gallop et al. (1994). Med Chem. 37:1233. Libraries of compounds may be presented in solution (e.g. Houghten (1992) Biotechniques 13:412-421), or on beads (Lam (199]) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91: 11422; Zuckermann et al. (1994), .J: Med. Chem. 37:2678; Cho et al. (1993), Science 261:1303; Carrell et al. (1994) Angew. Chem Int. Ed. Engl. 33:2059, or luciferase, and the enzymatic label detected by determination of conversion of an appropriate substrate to product.

In summary, based on the disclosure herein, those skilled in the art can develop tRNA-cognate aminoacyl-tRNA synthetase screening assays which are useful for identifying compounds which are useful for modulating aaRS-facilitated processes associated with its cognate tRNA priming function in retroviruses and more particularly LysRS-facilitated processes associated with tRNA^{Lys3} priming of RT in HIV. The assays of this invention may be developed for low-throughput, high-throughput, or ultra-high throughput screening formats.

The assays of this invention employ either natural or recombinant aaRS protein. Cell fraction or cell free screening assays for modulators of aaRS biological activity can use in situ, purified, or purified recombinant aaRS proteins. Cell based assays can employ cells which express aaRS protein naturally, or which contain recombinant aaRS gene constructs, which constructs may optionally include inducible promoter sequences. In all cases, the biological activity of aaRS can be directly or indirectly measured; thus modulators of aaRS biological activity can be identified. The modulators themselves may be further modified by standard combinatorial chemistry techniques to provide improved analogs of the originally identified compounds.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the detection of aminoacyl tRNA synthetases in HIV-1. Virions are pelleted from cell culture medium, and purified by centrifugation through sucrose onto a denser sucrose cushion. A. Western blots of aminoacyl tRNA synthetases found in the cytoplasm of HIV-1 transfected COS7 cells, and in the viruses produced from these cells. Western blots of cell lysates (C) or viral lysates (V) were probed with antibody to LysRS(a), IleRS(b), or ProRS(c). Numbers at the left of each panel represent molecular weight markers. B Resistance of viral-associated proteins to the protease subtilisin. Purified virions were either left untreated (N) or treated (S) with subtilisin. After subtilisin inactivation, viruses were lysed, and western blots of viral lysate were probed with antibodies to (a) CA; (b) gp120; or (c) LysRS. (d) Purified His₆-LysRS untreated or treated with subtilisin;

FIG. 2 shows the detection of LysRS in viruses purified by centrifugation through both sucrose and Optiprep gradients. Western blots of fractions from Optiprep gradients. A. Blot probed with anti-CA. V, sucrose-purified viral lysate before Optiprep gradient. B. Blot probed with anti-LysRS. K, purified His6-LysRS. C. Blot stained with commassie blue. M, marker proteins. D. Blot of pellet of material from cell culture media of non-transfected COS7 cells, probed with anti-LysRS;

FIG. 3 shows the detection of LysRS in cell lysates and lysates of sucrose-purified viruses produced from chronically infected cell lines. Western blots are probed with anti-LysRS. Cell lysates are from uninfected (−) or infected (+) cells. Numbers at the left represent molecular weight markers. LysRS, purified His₆-LysRS;

FIG. 4 shows the detection of LysRS in cell lysates and lysates of sucrose-purified viruses produced from COS7 cells transfected with HIV-1 DNA and a tRNA^{Lys3} gene. A. Western blot of viral lysate probed with anti-CA. wt, cells transfected with a plasmid containing wild type HIV-1 proviral DNA. Lys, cells transfected with a plasmid containing both wild type HIV-1 proviral DNA and a tRNA^{Lys3} gene. B. Western blot of cell lysate or viral lysate probed with anti-LysRS;

FIG. 5 shows the detection of LysRS in lysates of sucrose-purified viruses produced from COS7 cells transfected with wild type and mutant HIV-1 DNA. A. Western blot of viral lysate probed with anti-LysRS. LysRS, purified His₆-LysRS. wt, wild type. PR (−), viral protease-negative. P31L, substitution mutation in the region between the two Cys-His boxes in nucleocapsid. Dr2, insertion mutation in the connection domain of reverse transcriptase. Gag, Gag particles which do not contain Gag-Pol. P31L, Dr2, and Gag viral like particles do not selectively package tRNA^{Lys3}, while wt and PR (−) viruses do. COS7, cytoplasmic lysate. LysRS, purified His₆-LysRS. B. Western blot of viral lysate probed with anti-LysRS. Lanes: 2, wt; 3, P31L; 4,5, viruses from cells cotransfected with P31L DNA and DNA coding for wild type Gag-Pol (4) or wild type Gag (5); and

FIG. 6 shows the similarity of sequences between tRNA^{Lys1}, tRNA^{Lys2} and tRNA^{Lys3}.

FIG. 7 shows the effect of overexpression of wild type or mutant LysRS on the cytoplasmic concentration of LysRS. Western blot analysis of COS7 cell lysates, probed with either anti-LysRS (A) or anti-actin (B). Panel C shows the LysRS/Actin ratio determined from the data in panels A and B. Lane K, purified His-tagged human LysRS. The His₆-tagged human LysRS migrates more slowly than the large cytoplasmic LysRS species because of the N-terminal MRGSHHHHHHSSGWVD sequence appended to the full-length human LysRS used in these studies. The other lanes represent COS7 cells transfected with the following plasmids: 1, non-transfected; 2, pLysRS.F; 3, pLysRS.T; 4, BH10P-; 5, BH10P- and pLysRS.F; 6, BH10P- and pLysRS.T.

FIG. 8 shows the effect of overexpression of wild type or mutant LysRS on the viral concentration of LysRS. Western blot analysis of viral lysates probed with anti-LysRS (A) or anti-CA (B). Panel C shows the LysRS/Gag ratio determined from the data in panels A and B. Lane K, purified His-tagged human LysRS. The other lanes represent COS7 cells transfected with the following plasmids: 1, BH10P-; 2, BH10P- and pLysRS.F; 3, BH10P- and pLysRS.T.

FIG. 9 shows the effect of overexpression of wild type or mutant LysRS on the viral concentration of tRNA^{Lys}. A,B. Dot blots of total cellular (A) or viral (B) RNA were hybridized with with DNA probes to either -actin mRNA (A) or viral genomic RNA (B), and to tRNA^{Lys3} and tRNA^{Lys} (A,B). The ratios of tRNA^{Lys}/∃-actin mRNA (A) and tRNA^{Lys}/genomic RNA (B) in the cell and viral lysates, respectivel were determined for cells transfected with BH10P-, BH10P- and pLysRS.F, and BH10P- and pLysRS.T. C. 2D-PAGE patterns of viral tRNA extracted from virions containing wild type and mutant LysRS. Total viral RNA containing equal amounts of genomic RNA was labeled with the 3′-³²pCp end-labeling technique, and resolved by 2D PAGE. Viruses came from cells transfected with I, BH10P-, II, BH10P- and pLysRS.F, and III, BH10P- and pLysRS.T. Spot 3, tRNA^{Lys3}; Spots 1,2 tRNA^{Lys1,2}; Spot 4, tentatively identified as tRNA^{Asn}

FIG. 10 shows the interaction of wild type and mutant LysRS with tRNA^{Lys3} in vitro. Human tRNA^{Lys3} with 3′-end labeled with ³²pCp, and incubated in 20 ul binding buffer with wild type or mutant LysRS (truncation of N terminal 65 amino acids). Binding of LysRS to the tRNA^{Lys3} was analyzed by retardation of the electrophoretic mobility of tRNA^{Lys3} in native 6% 1 D PAGE. In each reaction tRNA^{Lys3} was 5 nM, while full length or truncated LysRS was present at uM concentrations of 1.5 uM (lanes 1,4), 0.3 uM (lanes 2, 5), or 0.06 uM (lanes 3,6). Mock, no LysRS.

FIG. 11 shows the distribution of wild type and mutant LysRS between nuclei and cytoplasm. COS7 cells were either non-transfected (−) or transfected with pLysRS.CF or pLysRS.CT. Cells were lysed in PBS buffer containing 0.1% Nonidet P-40 and 0.1% Triton X-100 as described in Materials and Methods. Nuclei were pelleted from the total cell lysate by centrifugation at 1000×g for 10 min, and the nuclear extract was prepared by lysing nuclei in RIPA buffer. Total cell lysate (T), nuclear extract (N), and the post-nuclear supernatant (C) were analyzed by western blotting. A. The distribution of endogenous LysRS in non-transfected cells (−), and of LysRS.CF and LysRS.CT in transfected cells. Endogenous LysRS is detected with anti-LysRS, while LysRS.CF and LysRS.CT are detected with anti-V5. B. A similar western blot as in (A), but probed with anti-tubulin. C. A similar western blot as in (A), but probed with anti-YYI, a nuclear transcription factor.

FIG. 12 shows the tRNA^{Lys3} structure. The tRNA^{Lys3} sequence is shown in cloverleaf form, and the anticodon mutant tRNA^{Lys3,}s created are shown, and listed as well.

FIG. 13 shows the expression of total tRNA^{Lys3} in cells and viruses. COS7 cells were transfected with a plasmid containing HIV-1 proviral DNA and a wild type or mutant tRNA^{Lys3} gene. Dot blots of cellular or viral RNA, containing equal amount of either ∃ actin mRNA (cellular RNA) or genomic RNA (viral RNA) were hybridized with a DNA probe complementary to the 3′ terminal 18 nucleotides of tRNA^{Lys3} to determine the total amount of tRNA^{Lys3} present in the cellular or viral RNA blots. (A,C). The top strip in (A) is a dot blot of increasing amounts of an in vitro tRNA^{Lys3} transcript, used to determine the linear standard curve shown in panel (C). The bottom two strips in panel (A) show dot blots of cellular or viral RNA isolated from cells transfected with HIV-1 proviral DNA and a wild type or mutant tRNA^{Lys3} gene. The results are plotted in panels B and D, respectively. A, cells transfected with HIV-1 DNA alone (BH10). B-F represent cells transfected with with HIV-1 DNA and tRNA^{Lys3} genes coding for the following anticodon sequence: B, UUU (wild type); C, CGA; D, CGU; E, UGU; F, UGA.

FIG. 14 shows the expression of specific wild type and mutant tRNA^{Lys3} in cells and viruses. COS7 cells were transfected with a plasmid containing HIV-1 proviral DNA and a wild type or mutant tRNA^{Lys3} gene. For each strip in panels A and B, the first portion contains dot blots of increasing amounts of an in vitro wild type or mutant tRNA^{Lys3} transcript, used to determine differences in efficiencies of hybridization for different anticodon probes. The following portion contains dot blots of cellular or viral RNA, containing equal amount of either 3 actin mRNA (cellular RNA) or genomic RNA (viral RNA), which were hybridized with a DNA probe complementary to anticodon arm of each wild type and mutant tRNA^{Lys3} so as to determine the amount of each tRNA^{Lys3} present in the cellular or viral RNA blots. These results are plotted in panel C (cellular) and panel D (viral). The controls in each strip in panel B is the wild type tRNA^{Lys3} in vitro transcript, to show that the anticodon probes do not detect wild type tRNA^{Lys3}. Panel A, cells transfected with HIV-1 DNA alone (A) or wild type tRNA^{Lys3} (B). Panel B. A-D represent cells transfected with with HIV-1 DNA and tRNA^{Lys3} genes coding for the following mutant anticodon sequence: A, CGA; B, CGU; C, UGU; D, UGA.

FIG. 15 shows the cytoplasmic expression of mutant tRNA^{Lys3}. COS7 cells were transfected with a plasmid containing HIV-1 proviral DNA and a wild type or mutant tRNA^{Lys3} gene, and differential centrifugation was used to separate nuclei and cytoplasm. Dot blots of the RNA extracted from the cytoplasmic fraction, representing equal amounts of 3 actin mRNA, were hybridized with with either the 3′ terminal DNA probe, which hybridizes to all tRNA^{Lys3,}s (A) or with anticodon probes specific for each mutant tRNA^{Lys3} (B-E). In panel A: 1, cells transfected with HIV-1 DNA alone (BH10). 2-5, cells transfected with HIV-1 DNA and tRNA^{Lys3} genes coding for the following anticodon sequence: 2, CGA; 3, UGA; 4, UGU; 5, UGA. In panels B-E: cells transfected with with HIV-1 DNA and tRNA^{Lys3} genes coding for the following anticodon sequence (lane 1): B, CGA; C, UGA; D, UGU; E, UGA. In each panel, C represents endogenous tRNA^{Lys3} in cells transfected with only HIV-1 DNA. Panel F: Western blot of nuclear and cytoplasmic fractions of transfected cells, numbered similarly to that in panel A. Blots probed with antibody to YY1, a transcription factor located in the nuclei. N, nuclear fraction; C, cytoplasmic fraction.

FIG. 16 shows the electrophoretic detection of acylated and deacylated tRNA^{Lys3}. Cellular RNA was isolated and amounts containing equal amounts of ∃ actin mRNA were electrophoresed under acidic conditions as described in the text. Northern blots of the cellular RNA were hybridized with with either the 3′ terminal DNA probe, which hybridizes to all tRNA^{Lys3,}s (A), or with anticodon probes specific for each mutant tRNA^{Lys3} (B-E). The first lane in each panel (1,8,11,14, and 17) represents a cellular RNA which was first exposed to alkaline pH to deacylate the tRNA (see Materials and Methods in Example 4). In panel A: 3, cells transfected with HIV-1 DNA alone (BH10). 2, and 4-7, cells transfected with with HIV-1 DNA and tRNA^{Lys3} genes coding for the following anticodon sequence: 2, UUU; 4, UGA; 5, UGU; 6, CGU; 7, CGA. In panels B-E: The middle lane represents the sample from cells transfected with with HIV-1 DNA and tRNA^{Lys3} genes coding for the following anticodon sequence: B, UGA; C, UGU; D, CGU; E, CGA. The last lane in each of these panels (10,13,16,19) represent RNA extracted from cells transfected only with HIV-1 proviral DNA. The aminoacylation results from lanes 1 and 2 in panel A, and the middle lanes in panels B-E are graphed in panel F.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

In view of the different protein-protein and protein-RNA interactions involved in ensuring that proper functioning of aminoacyl tRNA synthetase-processes associated with its cognate tRNA priming function occur, modulation of these processes can be effected in a number of ways, keeping in mind that 1) aminoacyl tRNA synthetase is the signal for its cognate tRNA packaging, 2) that the level of incorporation of tRNA packaging correlates with its aminoacylation; and 3) that cleavage of the synthetase which occurs in the virion may a) free the tRNA for annealing to the viral genomic RNA and b) cause deacylation of aminoacylated tRNA so that it can act as a primer for reverse transcription.

Herein, the relationship between viral tRNA^{Lys3} concentration and its placement onto the primer binding site (PBS) was analyzed by making use of naturally occurring variation in viral tRNA^{Lys3} concentration that we find in different virus preparations. The combination of tRNA^{Lys3} was artificially increased and decreased by the cytoplasmic synthesis of excess tRNA^{Lys3} or tRNA^{Lys2}, using cells transfected with plasmids coding for these tRNAs as well as for HIV-1. In virus from both transfected COS cells and infected cell lines, a direct correlation was found between viral tRNA^{Lys3} concentration, tRNA^{Lys3}-primed initiation of reverse transcription, and infectivity of the viral population.

During HIV-1 assembly, both tRNA^{Lys} and lysyl tRNA synthetase (LysRS) are incorporated into HIV-1. The LysRS is resistant to digestion with the protease subtilisin, and searches for two other amino acyl tRNA sythetases, ProRS and IleRS, revealed their absence in the virion. While the major cytoplasmic species of LysRS in infected cells has an Mr=70000 kd (large species), viral incorporation of tRNA^{Lys} is correlated with the packaging of an intermediate size LysRS species, Mr=63000 kd. This intermediate species is the major form of LysRS found in virions produced from chronically infected cells (H9, U937, PLB, CEMss), while in wild type or protease-negative HIV-1 produced from COS cells (HIV(COS)), both the large and intermediate LysRS species are found. The presence of the intermediate size LysRS in protease-negative viruses indicates that a cellular protease is involved. The intermediate LysRS species becomes the major LysRS species in HIV(COS) when viral tRNA^{Lys3} packaging is increased as a result of a cotransfection of COS cells with HIV-1 proviral DNA and a tRNA^{Lys3} gene. In mutant HIV(COS) which are defective in tRNA^{Lys3} packaging (P31L(NC mutation), Dr2 (RT mutation), and Gag-only particles), no intermediate size LysRS species is detected. Rescue of tRNA^{Lys3} packaging in the P31L mutant with wild type Gag-Pol also results in an increase in the incorporation of the intermediate form of LysRS within the virus.

tRNA^{Lys} packaging in HIV is shown herein to be limited by LysRS, since the overproduction of LysRS from a cotransfected plasmid encoding LysRS results in up to a 2 fold increase in a) the incorporation of both tRNA^{Lys} isoacceptors into the viruses, b) increased placement on the viral genome, and c) increased viral infectivity. Overproduction of a mutant LysRS lacking the N terminal 65 amino acids also results in increases in LysRS viral packaging, but no increase in tRNA^{Lys} viral packaging is observed, since the mutant LysRS cannot bind to tRNALys.

The present invention is illustrated in further detail by the following non-limiting examples.

### EXAMPLE 1

### Correlation Between the Viral tRNA Concentration in the HIV Virion, the Level of Initiation of Reverse Transcriptase and HIV Infectivity

During retroviral assembly, particular species of cellular tRNA are selectively packaged into the virus, where they are placed onto the primer binding site (PBS) of the viral genome, and are used to initiate the reverse-transcriptase-catalyzed synthesis of minus strand cDNA. tRNA^{Trp} is the primer for all members of the avian sarcoma and leukosis virus group examined to date (Faras et al., 1975; Harada et al., 1975; Peters et al., 1980; Sawyer et al., 1973; Waters et al., 1977; Waters et al., 1975). The common primer tRNAs in mammalian retroviruses are tRNA^{Pro} and tRNA^{Lys}. tRNA^{Pro} is the common primer for Murine Leukemia Virus (MuLV) (Harada et al., 1979; Peters et al., 1977; Taylor et al., 1977). In mammalian cells, there are three major tRNA^{Lys} isoacceptors (Raba et al., 1979). tRNA^{Lys1,2}, representing two tRNA^{Lys} isoacceptors differing by one base pair in the anticodon stem, is the primer tRNA for several retroviruses, including Mason-Pfizer Monkey virus (MPMV) and Human Foamy Virus (HFV) (Leis et al., 1993). tRNA^{Lys3} serves as the primer for Mouse Mammary Tumor Virus (Peters et al., 1980; Waters et al., 1978), and the lentiviruses such as Equine Infectious Anemia Virus (EIAV), Feline Immunodeficiency Virus (FIV), Simian Immunodeficiency Virus (SIV), Human Immunodeficiency Virus type 1 (HIV-1), and Human Immunodeficiency Virus type 2 (HIV-2) (Leis et al., 1993).

Selective packaging of primer tRNA is defined as an increase in the percentage of the low molecular weight RNA population representing primer tRNA in moving from the cytoplasm to the virus. For example, in AMV, the relative concentration of tRNA^{Trp} changes from 1.4% in the cytoplasm to 32% in the virus (Waters et al., 1977). In HIV-1 produced from COS7 cells transfected with HIV-1 proviral DNA, both primer tRNA^{Lys3} and tRNA^{Lys1,2} are selectively packaged, and the relative concentration of tRNA^{Lys} changes from 5-6% to 50-60% (Mak et al., 1994). Both tRNA^{Lys3} and tRNA^{Lys1,2} are packaged into HIV-1 with equal efficiency since the tRNA^{Lys3}:tRNA^{Lys1,2} ratio in the virus reflects the cytoplasmic ratio, even when the cytoplasmic ratio is altered (Huang et al., 1994). In AKR Murine Leukemia Virus (AKR-MuLV), selective packaging of primer tRNA^{Pro} is less dramatic, going from a relative cytoplasmic concentration of 5-6% to 12-24% of low molecular weight RNA (Waters et al., 1977). Selective packaging of primer tRNA occurs independently of viral genomic RNA packaging in MuLV, HIV-1, and Avian Sarcoma Virus (Levin et al., 1979; Mak et al., 1994; Prats et al., 1988), and has been shown in HIV-1 to occur independently of Gag and Gag-Pol processing (Khorchid et al., 2000; Mak et al., 1994). Selective packaging of primer tRNAs suggests that the increase in viral concentration of these tRNAs may facilitate the placement of the tRNA onto the PBS. This may be the case for avian retroviruses (Fu et al., 1997; Peters et al., 1980) and HIV-1 (Mak et al., 1994), but is apparently not the case for MuLV, where mutations in RT which prevent tRNA^{Pro} packaging do not inhibit its placement on the genome (Fu et al., 1997; Levin et al., 1984; Levin et al., 1981). Experiments with RT(−) mutants in avian retroviruses and in HIV-1 do not make clear as to whether reduced genomic placement of primer tRNA is due to the reduction of primer tRNA in the virus or to the absence of functional RT sequences required to place the tRNA on the genome. However, recent experiments have shown that while Pr160^{gag-pol} is required for selective packaging of tRNA^{Lys3} into Pr55^{gag} particles (Mak et al., 1994), Pr55^{gag} plays a major role in placing tRNA^{Lys3} onto the PBS (Cen et al., 1999; Feng et al., 1999).

Materials and Methods

Plasmid Construction

SVC21BH10 is a simian virus 40-based vector containing wild-type HIV-1 proviral DNA. SVC21BH10-^{Lys3} and SVC21 BH10-^{Lys2} contain both wild-type HIV-1 proviral DNA and a human tRNA^{Lys3} or tRNA^{Lys2} gene, respectively. These vectors were constructed as previously described (Huang et al., 1994).

Virus Infection/Transfection and Purification

COS7 cells were transfected using the calcium phosphate method as previously described (Mak et al., 1994). Supernatant was collected 63 hours post-transfection. For H9, CEMSS, PLB and U937, an equal amount of infected and non-infected cells (5×10⁶ cells each) were mixed together, and supernatant containing virus was collected 3 days post-infection. Virus from all cell types was pelleted from culture medium by centrifugation in a Beckman Ti45 rotor at 35,000 rpm for 1 hour. The viral pellets were then purified by centrifugation in a Beckman SW41 rotor at 26,500 rpm for 1 hour through 15% sucrose onto a 65% sucrose cushion. The band of purified virus was removed and pelleted in 1×TNE in a Beckman Ti45 rotor at 40,000 rpm for 1 hour. Viral genomic RNA was extracted using guanidium isothiocynate, as previously described (Jiang et al., 1993).

1D and 2D Page

Electrophoresis of ³²pCp-labelled viral RNA was carried out at 4° C. with the Hoeffer SE620 gel electrophoresis apparatus. The gel size was 14 by 32 cm. The first dimension was run in an 11% polyacrylamide-7M urea gel for 16 hours at 800 V. After autoradiography, the piece of gel containing RNA was cut out, and run for 30 hours (25 Watt limiting); this was followed by autoradiography. All electrophoretic runs were carried out in 0.5×TBE (1×TBE is 50 mM Tris, 5 mM boric acid, 1 mM EDTA-Na₂). The electrophoretic gel patterns shown in this paper show only low molecular weight RNA, since the high-molecular weight viral genomic RNA cannot enter into the polyacrylamide gels. Furthermore, these patterns represent only the most abundant tRNA species present, since longer film exposures will reveal the presence of more minor-abundance species.

Packaging of tRNA^{Lys3}

The relative amount of tRNA^{Lys3} per copy of HIV-1 genomic RNA was determined by dot blot hybridization. Each sample of total viral RNA was blotted onto Hybond N+l nylon membranes (Amersham Pharmacia) in triplicate, and was probed with a 5′³²P-end-labelled 18-mer DNA probe specific for the 3′ end of tRNA^{Lys3} (5′-TGGCGCCCGMCAGGGAC-3′). The relative amounts of tRNA^{Lys3} per sample were analyzed using phosphor-imaging (BioRad). The blots were then stripped according to the manufacturer's instructions, and were re-probed with a 5′³²P-end-labelled 17-mer DNA probe specific for the for the 5′ end of HIV-1 genomic RNA, upstream of the primer binding site (5′-CTGACGCTCTCGCACCC-3′). Phosphor-imaging was used to quantitate the relative amount of HIV-1 genomic RNA per sample, and the relative amount of tRNA^{Lys3} per copy of HIV-1 genomic RNA was determined.

Primer Extension

tRNA^{Lys3}-primed initiation of reverse transcription was measured by the ability of tRNA^{Lys3} to be extended by 6 bases in an in vitro HIV-1 reverse transcription reaction. For each sample, equal amounts of total viral RNA (5×10⁸ copies of genomic RNA, measured as previously described (Huang et al., 1994)) were used as a source of primer tRNA/template. The sequence of the first 6 deoxynucleoside triphosphates incorporated is CTGCTA. The reactions were carried out in a volume of 20 μL containing 50 mM Tris-HCl (pH 7.8), 100 mM KCl, 10 mM MgCl₂, 10 mM DTT, 0.2 mM dCTP, 0.2 mM dTTP, 5 μCi α-³²P-dGTP and 0.05 mM ddATP (instead of dATP, thereby terminating the reaction at 6 bases), 50 ng HIV-1 RT, and RNase inhibitor (Amersham Pharmacia). After incubation for 15 minutes at 37° C., the samples were precipitated with isopropanol, and were electrophoresed in a 6% polyacrylamide gel at 70 W for 1.5 hours. Relative amounts of tRNA^{Lys3} placement were analyzed by comparing the intensity of bands with phosphor-imaging.

Viral Infectivity

Viral infectivity was measured by the MAGI assay (Kimpton et al., 1992). MAGI cells are CD4+ HeLa cells containing an HIV-1 LTR fused to a β-galactosidase reporter gene. A total of 4×10⁴ cells per well were cultured in 1 mL of media, in 24-well plates. After 24 hours, the media was removed and was replaced with 150 μL of culture medium containing various dilutions of virus. DEAE-Dextran was added to a final concentration of 20 μg/ml, and viral absorption took place for 2 hours, after which 1 mL of fresh culture medium was added. 48 hours later, the medium was removed and fixative (1% formaldehyde, 0.2% gluteraldehyde in PBS) was added for 5 minutes. The fixative was removed and 200 μL of staining solution was added (for 1 mL: 950 μL PBS, 20 μL of 0.2 M potassium ferrocyanide, 20 μL of 0.2 M potassium ferricyanide, 1.0 μL of 2 M MgCl₂, and 10 μL of X-gal stock [stock=40 mg/mL in DMSO]). The cells were washed twice with PBS and the number of blue cells per well per equal amount of p24 were counted.

Protein Analysis

Viral particles were purified as described above, and viral proteins Were extracted with RIPA buffer (10 mM Tris, pH 7.4, 100 mM NaCl, 1% sodium deoxycholate, 0.1% SDS, 1% NP40, 2 mg/ml aprotinin, 2 mg/ml leupeptin, 1 mg/ml pepstatin A, 100 mg/ml PMSF). The viral lysates were analyzed by SDS PAGE (10% acrylamide), followed by blotting onto nitrocellulose membranes (Amersham Pharmacia). Detection of protein by Western blotting utilized monoclonal antibodies that are specifically reactive with HIV-1 capsid (Zepto Metrocs Inc.) and reverse transcriptase (a kind gift from M. Parniak, Montreal, Canada). Detection of HIV proteins was performed by enhanced chemiluminescence (NEN Life Sciences Products) using sheep anti-mouse as a secondary antibody (Amersham Life Sciences).

Results

Effect of Natural Variation of tRNA^{Lys3} Packaging into HIV-1 (COS) upon the Initiation of Reverse Transcription and Viral Infectivity

Table 1A lists the tRNA^{Lys3}/genomic RNA ratio for 7 different preparations of HIV-1 produced from COS7 cells. The values are normalized to the viral preparation containing the highest ratio, i.e. COS7a. Each value listed is the average of experiments done in triplicate, in which dot blots of total viral RNA were hybridized with radioactive DNA probes complementary to either tRNA^{Lys3} or genomic RNA. It can be seen that within this sampling, the tRNA^{Lys3}/genomic RNA ratio can vary as much as three fold.

Three other viral preparations, COS7A, COS7B, and COS7C, are listed in Table 1 B. Normalizing against COS7B, the relative tRNA^{Lys3}/genomic RNA ratios are, respectively, 0.74, 1.00, and 0.52. We have previously shown that alterations in the viral concentration of tRNA^{Lys3} is reflected in opposite alterations in the viral concentration of tRNA^{Lys1,2}, i.e., an increase in the viral concentration of one isoacceptor results in a decrease in the viral concentration of the other isoacceptor (Feng et al., 1999). 2 dimension polyacrylamide gel electrophoresis (2D PAGE) patterns of low molecular weight viral RNA in these preparations, confirms this to be so. The identity of the tRNA^{Lys} isoacceptors found in each spot have been previously determined (Frugier et al., 2000). Analysis of the relative densities of each spot by phosphor-imaging gives the tRNA^{Lys3}/tRNA^{Lys1,2} ratio for each preparation. These are listed in Table 1B, and it can be seen that they correlate with the tRNA^{Lys3}/genomic RNA ratios. The changes in viral tRNA^{Lys3} concentrations are not as large as the corresponding changes in tRNA^{Lys3}/tRNA^{Lys1,2} ratios, because the ratios are determined by opposing changes in both tRNA^{Lys3} and tRNA^{Lys1,2} viral concentrations.

TABLE 1
A

| Virus Sample from | a | b | c | d | e | F | g |
|---|---|---|---|---|---|---|---|
| COS7 | | | | | | | |
| Relative concentration | 1.00 | 0.33 | 0.61 | 0.41 | 0.32 | 0.80 | 0.65 |
| of tRNA^{Lys3}/ | | | | | | | |
| genomic RNA* | | | | | | | |

*normalized to COS7a

B

| | Relative | Ratio | Relative | Relative |
|---|---|---|---|---|
| Virus sample | concentration of | tRNA^{Lys3} | amount of | infectivity |
| produced | tRNA^{Lys3} per | to | tRNA^{Lys3} | (blue cells/ |
| from COS7 | genomic RNA* | tRNA^{Lys1,2*} | extension* | p24)* |
| COS7A | 0.74 | 0.36 | 0.67 | 0.80 |
| COS7B | 1.00 | 1.97 | 1.00 | 1.00 |
| COS7C | 0.52 | 0.52 | 0.60 | 0.57 |

*normalized to COS7B

We next investigated in these three viral preparations whether the amount of tRNA^{Lys3} packaged into the virus reflects the amount of extendable tRNA^{Lys3} placed onto the primer binding site (PBS). The first 6 bases incorporated into DNA during the initiation of reverse transcription are CTGCTA. tRNA^{Lys3} extension was measured in an in vitro reaction using equal amounts of genomic RNA, exogenous HIV-1 RT, dCTP, dTTP, α-³²P-dGTP, and ddATP. This will result in a six base extension of the tRNA^{Lys3}, and the amount of DNA extension/genomic RNA was determined on 1 D-PAGE (data not shown). Relative signal intensities were measured by phosphor-imaging, the results of which are listed in Table 1B. This data indicates a correlation between tRNA^{Lys3} incorporated into the virus and the amount of extendable tRNA^{Lys3} placed onto the PBS.

The relative infectivity of the three viral preparations was also measured using the MAGI assay (Huang et al., 1997), which measured single round infectivity. CD4-positive Hela cells containing the β-galactosidase gene fused to the HIV-1 LTR are infected with virus. Cells infected with HIV-1 will have the β-galactosidase gene expressed, and such cells can be detected using an appropriate substrate for the enzyme, such as X-gal, whose metabolism turns the cells blue. The number of blue cells is a measure of viral infectivity. As indicated in Table 1B, the relative infectivity of the different viral populations is directly correlated with tRNA^{Lys3} packaging and extension.

Effect of Artificially Altering the tRNA^{Lys3} Concentration in HIV-1 (COS) upon Initiation of Reverse Transcription and Viral Infectivity.

We have previously shown that viral tRNA^{Lys3} content can be increased by transfecting COS7 cells with an SV40-based plasmid containing both the HIV-1 proviral DNA and a human tRNA^{Lys3} gene, and that as a result, tRNA^{Lys1,2} packaging into the virus decreases (Huang et al., 1994). Herein, we have measured the effect of this artificial increase in viral tRNA^{Lys3} (virus BH10-^{Lys3} in Table 2) upon tRNA^{Lys3}-primed initiation of reverse transcription and viral infectivity. We have also, in a similar manner, produced viruses with an excess of tRNA^{Lys2} and a decrease in viral tRNA^{Lys3} (virus BH10-^{Lys2} in Table 2), by transfecting COS7 cells with a plasmid containing the HIV-1 proviral DNA and a human gene for tRNA^{Lys2} (obtained from Dr Robert M. Pirtle, University of North Texas). The relative concentration of tRNA^{Lys3}/virion, normalized to wild type, was determined as above, by hybridizing dot blots of total viral RNA with DNA probes specific for tRNA^{Lys3} and for genomic RNA, and values are listed in Table 2. BH10-Lys3 has approximately 1.6 times more tRNA^{Lys3} than wild type, while BH10-Lys2 has less than one fifth the amount of tRNA^{Lys3} found in wild type virions. The 2D PAGE pattern for low molecular weight RNA in wild type HIV-1 (BH10), BH10-Lys3, and BH10-Lys2 was assessed (data not shown), and the tRNA^{Lys3}/tRNA^{Lys1,2} ratios determined by phosphor-imaging of these gels are listed in Table 2. BH10-Lys3 has an additional small dark spot which has been identified as an additional tRNA^{Lys3} by a partial T1 digestion pattern (data not shown) identical to the partial T1 digestion pattern of the major tRNA^{Lys3} spot (Jiang et al., 1993). This species can sometimes be seen as a very light spot in wild type virus. As found above for the different wild type HIV(COS), the changes in viral tRNA^{Lys3} concentrations are not as large as the corresponding changes in tRNA^{Lys3}/tRNA^{Lys1,2} ratios because the ratios are determined by opposing changes in both tRNA^{Lys3} and tRNA^{Lys1,2} viral concentrations.

TABLE 2

| | Relative | Ratio | Relative | Relative |
|---|---|---|---|---|
| Virus sample | concentration of | TRNA^{Lys3} | amount of | infectivity |
| produced | tRNA^{Lys3} per | to | tRNA^{Lys3} | (blue cells/ |
| from COS7 | genomic RNA* | tRNA^{Lys1,2*} | extension* | p24)* |
| BH10 | 1.00 | 0.54 | 1.00 | 1.00 |
| BH10-Lys3 | 1.56 | 28.0 | 1.89 | 2.63 |
| BH10-Lys2 | 0.17 | 0.04 | 0.36 | 0.42 |

*normalized to BH10

As described above for wild type HIV (COS), we measured the ability of the placed tRNA^{Lys3} from each viral preparation to be extended 6 bases in an in vitro reverse transcription reaction. The amount of tRNA^{Lys3} extension/genomic RNA was determined on 1 D-PAGE, (data not shown). Relative signal intensities were analyzed by phosphor-imaging, the results of which are listed in Table 2. This data indicates a direct correlation between tRNA^{Lys3} incorporated into the virus and the amount of extendable tRNA^{Lys3} placed onto the PBS. The relative infectivity of these different viral populations was also measured by the MAGI assay, and as indicated in Table 2, higher infectivity is associated with greater tRNA^{Lys3} packaging and initiation of reverse transcription.

While this data indicates that initiation of reverse transcription mirrors tRNA^{Lys3} concentration in the virus, an alternative interpretation is that packaging and genomic placement of tRNA^{Lys3} are both independently influenced by the packaging of Pr160^{gag-pol}. We therefore looked at the RT/p24 ratios in BH10-Lys3 and BH10-Lys2. A Western blot of total viral protein from these two virus types probed with antibody to either p24 (anti-CA) or to RT (anti-RT) was carried out (data not shown). The ratio of RT/p24, determined by phosphor-imaging, is 2.81 and 2.63, respectively for BH10-Lys3 and BH10-Lys2, making it unlikely that the five fold difference in placement of extendable tRNA^{Lys3} between these two virus types is due to increased incorporation of Pr160^{gag-pol}.

Effect of Natural Variation of tRNA^{Lys3} Packaging into HIV-1 Produced in Chronically Infected Cell Lines upon the Initiation of Reverse Transcription and Viral Infectivity.

The natural variation in tRNA^{Lys3} packaging in HIV-1 (COS) is also found in HIV-1 produced in chronically infected cell lines. Table 3A lists the tRNA^{Lys3}/genomic RNA ratio in HIV-1 produced from 4 different chronically infected cell lines, and from transfected COS7 cells. Two different viral preparations were used for each cell type, and the values were normalized to the the viral preparation containing the highest ratio, ie, COS7b. Each value listed is the average of experiments done in triplicate, in which dot blots of total viral RNA were hybridized with radioactive DNA probes complementary to either tRNA^{Lys3} or genomic RNA.

In Table 3B, using different viral preparations, we measured the correlation between viral tRNA^{Lys3} concentration, tRNA^{Lys3} extension by RT, and viral infectivity, using methods described above for measuring these parameters in transfected COS7 cells. In Table 3B, we see that the relative amount of tRNA^{Lys3} extension and viral infectivity are directly correlated with the amount of viral tRNA^{Lys3} packaging.

TABLE 3
A

| Cell source of | Sample | | | | | |
|---|---|---|---|---|---|---|
| HIV-1 RNA | @ | H9 | CEMSS | PLB | U937 | COS7 |
| Relative | A | 0.47 | 0.45 | 0.43 | 1.16 | 0.43 |
| concentration | B | 0.94 | 0.33 | 0.13 | 0.36 | 1.00 |
| of | | | | | | |
| tRNA^{Lys3}/geno | | | | | | |
| mic RNA* | | | | | | |

*normalized to COS7 b

B

| | Relative | | |
|---|---|---|---|
| | concentration of | Relative amount | Relative |
| Cell source of | tRNA^{Lys3} per | of tRNA^{Lys3} | infectivity |
| HIV-1 | genomic RNA* | extension* | (blue cells/p24)* |
| H9 | 0.54 | 0.60 | 0.53 |
| CEMSS | 1.00 | 1.00 | 1.00 |
| PLB | 0.43 | 0.66 | 0.23 |
| U937 | 0.47 | 0.60 | 0.36 |

*normalized to CEMSS

Stability of Variation in tRNA^{Lys3} Packaging

To further understand the nature of the variation in tRNA^{Lys3} packaging, we examined its stability in H9 cells chronically infected with HIV-1. Every 3 days, cultures were supplemented with fresh uninfected H9 cells, keeping the cell concentration constant at 1.0×10⁶ cells/ml, and viruses were harvested at 3 days, 2 weeks, one month, and 2 months. 2D PAGE patterns of low molecular weight RNA taken from these viruses enabled a determination of the ratios of tRNA^{Lys3}/tRNA^{Lys1,2}, phosphor-imaging. Taken together, we have shown that increases in the ratio of tRNA^{Lys3}/tRNA^{Lys1,2} are correlated with increases in tRNA^{Lys3} packaging into the virus and that over a two month period, the tRNA^{Lys3}/tRNA^{Lys1,2} changes, but that such changes are not stable.

Discussion

The work herein indicates a direct relationship between tRNA^{Lys3} incorporated into the virus, tRNA^{Lys3}-primed initiation of reverse transcription, and infectivity of the viral population. Is placement proportional to the number of tRNA^{Lys3} molecules within a virion, or are we simply recruiting new virions in the population that previously did not contain any tRNA^{Lys3}? The existence of defective viruses containing no tRNA^{Lys3} is unlikely. We have shown that in a homogeneous population of HIV-1 RT(−) mutants which are defective in selective tRNA^{Lys} packaging, there is still an average of 1-2 molecules tRNA^{Lys3} packaged randomly per virion (Mak et al., 1994; Mak et al., 1997). Furthermore, tRNA^{Lys3} extension in these RT mutant populations is defective (10% wild type (Mak et al., 1994) and unpublished results). Rather than 10% of the defective viruses packaging all the tRN^{Lys}, it seems more likely that all or nearly all virions in this defective RT(−) population contain 1-2 molecules of tRNA^{Lys3}, and that this is not sufficient for correct placement of even one of the two PBS sequences present in each virion.

If the increased tRNA^{Lys3} packaging is accompanied by increased Pr160^{gag-pol} incorporation, and if this viral protein is involved in packaging of tRNA^{Lys} into the virus, the increase in this protein could be responsible for greater tRNA^{Lys3} placement. This seems unlikely for several reasons. First, we have previously shown that increased packaging of one tRNA^{Lys} isoacceptor family results in the reduction of the other tRNA^{Lys} isoacceptor family (Huang et al., 1.994), something also seen by 2D-PAGE analysis (data not shown). The total number of tRNA^{Lys} molecules in the virus does not change significantly, so there is no reason to assume that increased tRNA^{Lys3} packaging is accompanied by an increased packaging of Pr160^{gag-pol}. This was in fact demonstrated by western blots of protein from BH 10-Lys3 and BH10-Lys2 that the RT/p24 ratios are similar, even though tRNA^{Lys3} extension in BH10-Lys2 is only 20% that found in BH10-Lys3 (data not shown). Secondly, work has shown, both in vitro (Feng et al., 1999) and in vivo (Cen et al., 1999), that the main viral protein involved in annealing tRNA^{Lys3} to the PBS is Pr55^{gag}, and not Pr160^{gag-pol}. It is therefore likely that the inability of RT(−) mutants in avian retroviruses and HIV-1 to place primer tRNA onto the PBS is due to the inability of these mutants to package primer tRNA, which does require intact RT sequence within Pr160^{gag-pol}, and is not due to the absence of functional RT sequences in the virion. Interestingly, this correlation between primer tRNA packaging and placement has not been found in RT(−) MuLV, i.e., RT(−) mutants which reduce packaging of primer tRNA^{Pro} do not reduce primer tRNA^{Pro} placement on the PBS (Fu et al., 1997). Since Gag, rather than Gag-Pol, has been found to be sufficient for primer tRNA placement in vitro (Feng et al., 1999), the insensitivity of genomic placement of primer tRNA in MuLV to viral concentration of primer tRNA may reflect an increased binding affinity between murine Gag and tRNA^{Pro} compared to the binding affinity between HIV-1 Gag and tRNA^{Lys3} or avian Gag and tRNA^{Trp}. This would also explain why the selective incorporation of primer tRNA in wild type virions is not required to be as strong in MuLV as in avian retroviruses or HIV-1.

The variation in tRNA^{Lys3} packaged/virion that we report here was not previously seen in our earlier work with HIV-1-transfected COS cells (Huang et al., 1994; Mak et al., 1997). What is responsible for the variability in the viral tRNA^{Lys3} concentration? Since cultures of chronically infected cell lines are producing viruses which are constantly infecting uninfected cells, mutations in viral genes might occur over time during reverse transcription, and account for variability in tRNA^{Lys} packaging. However, since the variation in tRNA^{Lys3} packaging is not stable this does not seem to be occurring. The inability of the virus to maintain higher levels of tRNA^{Lys3} packaging, which we have shown to be associated with higher infectivity rates, also indicates that other constraints exist which must prevent viral mutations which would lead to higher tRNA^{Lys3} packaging. COS3 cell transfection studies also indicate that the variability is not due to mutation in viral genes since reverse transcription is not involved in producing virions in this system. While the variability of tRNA^{Lys3} packaging in such viruses could be due to errors arising during RNA transcription, this would also seem unlikely to have a significant effect upon the whole population of first round viruses. The most likely explanation for the existence of unstable variation in the tRNA^{Lys3} packaging is that it is due to an unstable variation in the cell environment. This could result in variations in the tRNA^{Lys3} concentration in the cytoplasm, which previous work (Huang et al., 1994) and the work herein with BH10-Lys3 and BH10-Lys2 have shown to have a direct effect upon the amount of tRNA^{Lys3} packaged. The fact that variations detrimental to other events in the viral life cycle do not mask the increases in viral infectivity associated with increased tRNA^{Lys3} packaging and placement indicate that the variation in tRNA^{Lys3} packaging may represent a rather unique cellular event affecting viral infectivity, perhaps because tRNA^{Lys} is one of the few cellular factors known to be required in the viral life cycle.

### EXAMPLE 2

### Incorporation of Lysyl-tRNA synthetase into HIV-1

During HIV-1 assembly, the major cellular tRNA^{Lys} isoacceptors, tRNA^{Lys1,2} and tRNA^{Lys3} are selectively packaged into the virus (Jiang et al., 1993), and tRNA^{Lys3} is used as the primer for the reverse transcriptase-catalyzed synthesis of minus strand DNA (Leis et al., 1993). The selective packaging of tRNA^{Lys} into HIV-1 occurs independently of both genomic RNA packaging (Jiang et al., 1993) and the processing of the viral precursor proteins Pr55^{gag} and Pr160^{gag-pol} (Mak et al., 1994), but does depend on the participation of both of these unprocessed proteins. While Pr55^{gag} alone is sufficient to form viral particles, and binds to both viral genomic RNA (Berkowitz et al., 1996) and Pr160^{gag-pol} (Park et al., 1992; Smith et al., 1993), it is not known if a specific binding of Pr55^{gag} to tRNA^{Lys} contributes to tRNA^{Lys} selective packaging. Evidence for an interaction between Pr55^{gag} and tRNA^{Lys3} comes not from tRNA^{Lys3} packaging studies, but from tRNA^{Lys3} placement studies which indicate that this protein, and not Pr160^{gag-pol}, plays a major role in annealing tRNA^{Lys3} onto the PBS in vitro (Feng et al., 1999) or in vivo (Cen et al., 1999).

In considering the interactions involved between viral proteins and tRNA^{Lys} during packaging, it must be taken into account that tRNAs have been reported to be channeled from one component of the translational machinery to the next, and thus, may never be free of this synthetic machinery (Stapulionis et al., 1995). Such components could involve ribosomes, elongation factors, and aminoacyl-tRNA synthetases (aaRSs). Although it has been shown that elongation factor-1 alpha is packaged into HIV-1 via an interaction with Pr55^{gag} (Cimarelli et al., 1999), it is not clear how this protein, which binds to all aminoacylated tRNAs, would confer the ability to selectively package tRNA^{Lys} into the virion. Another tRNA-binding protein in the cytoplasm which is more specific for tRNA^{Lys} is lysyl-tRNA synthetase (LysRS). This enzyme is an attractive candidate for interacting specifically with viral proteins, and may play a role in the transport of the three tRNA^{Lys} isoacceptors into the virions.

We show herein that the tRNA^{Lys}-binding protein, lysyl-tRNA synthetase (LysRS), is also selectively packaged into HIV-1. The viral precursor protein Pr55^{gag} alone will package LysRS into Pr55^{gag} particles, independently of tRNA^{Lys}. With the additional presence of the viral precursor protein Pr160^{gag-pol}, tRNA^{Lys} and LysRS are both packaged into the particle. While the predominant cytoplasmic LysRS has an apparent Mᵣ=70,000, viral LysRS associated with tRNA^{Lys} packaging is shorter, with an apparent Mᵣ=63,000. The truncation occurs independently of viral protease, and might be required to facilitate interactions involved in the selective packaging and genomic placement of primer tRNA^{Lys3}.

Materials and Methods

Plasmid Construction

SVC21.BH10 is a simian virus 40-based vector that contains full-length wild-type HIV-1 proviral DNA and was a gift from E. Cohen, University of Montreal. pSVGAG-RRE-R and pSVFS5TprotD25G, which code for either Gag or unprocessed GagPol, respectively, have been described previously (Smith et al., 1990; Smith et al., 1993). Viral production from either of these two plasmids, which contain the Rev response element (RRE), requires co-transfection with a Rev protein expression vector, such as pCMV-REV. Thus, co-transfection of pSVGAG-RRE-R with pCMV-REV is required to produce virus-like particles containing unprocessed Pr55^{gag} precursor protein. In this report, pSVSF5TprotD25G is co-transfected with SVC21P31L, a plasmid coding for HIV-1 proteins including Gag and Rev, but not for stable GagPol. The construction of the mutants SVC21Dr2, and SVC21P31L have been described previously (Huang et al., 1997; Mak et al., 1997).

Cell Lines

COS7 cells were maintained in Dulbecco modified Eagle medium with 10% fetal bovine serum and antibiotic. H9, PLB, CEMss and U937 cell lines (+/−, infected or non-infected) were grown in RPMI1640 with 10% fetal bovine serum and antibiotic.

Production of Wild-Type and Mutant HIV-1 Virus

Transfection of COS7 cells with the above plasmids by the calcium phosphate method was as previously described (Mishima et al., 1995). Viruses were isolated from COS7 cell culture medium 63 h posttransfection, or from the cell culture medium of infected cell lines. The virus-containing medium was first centrifuged in a Beckman GS-6R rotor at 3,000 rpm for 30 minutes and the supernatant was then filtered through a 0.2 μm filter. The viruses in the filtrate were then pelleted by centrifugation in a Beckman Ti45 rotor at 35,000 rpm for 1 h. The viral pellet was then purified by centrifugation with a Beckman SW41 rotor at 26,500 rpm for 1 h through 15% sucrose onto a 65% sucrose cushion.

Western Blotting

Sucrose-gradient-purified virions were resuspended in 1× radioprecipitation assay buffer (RIPA buffer: 10 mM Tris, pH 7.4, 100 mM NaCl, 1% deoxycholate, 0.1% sodium dodecyl sulfate (SDS), 1% Nonidet P-40, protease inhibitor cocktail tablets (Boehringer Mannheim)). Western blot analysis was performed using either 300 μg of cellular protein or 10 μg of viral protein, as determined by the Bradford assay (Bradford et al., 1976). The cellular and viral lysates were resolved by SDS-PAGE followed by blotting onto nitrocellulose membranes (Gelman Sciences). Detection of protein on the Western blot utilized monoclonal antibodies or antisera specifically reactive with viral p24 and gp120, as well as with different aminoacyl-tRNA synthetases. Mouse anti-p24 and rabbit anti-gp120 were purchased from Intracel Corp. Rabbit anti-LysRS, anti-ProRS, and anti-IleRS were isolated following three subcutaneous injections of purified protein with 3-4 weeks intervals between injections (150-300 μg total protein). An N-terminal truncated form of human LysRS (Shiba et al., 1997), and a C-terminal truncated form of human IleRS (Shiba et al., 1994) were used in these preparations. The complete amino acid sequence of human LysRS can be found for example in Shiba et al., 1997 as well as in Genbank under accession number D32053. Human ProRS is derived from the C-terminal domain (amino acid residues 926-1440) of human glutamyl-prolyl-tRNA synthetase (GluProRS), and was purified as described (Heacock et al., 1996). Western blots were analyzed by enhanced chemiluminescence (ECL kit, Amersham Life Sciences) using goat anti-mouse or donkey anti-rabbit (Amersham Life Sciences) as a secondary antibody. The sizes of the detected protein bands were estimated using pre-stained high molecular mass protein markers (GIBCO/BRL).

OptiPrep [symbol] Gradient

Virions were sometimes purified by replacing centrifugation through sucrose with centrifugation in an OptiPrep [symbol] velocity gradient (60% [wt/vol] iodixanol, Life Technologies). Iodixanol gradients were prepared in PBS as 11 steps in 1.2% increments ranging from 6 to 18%. Virions were layered onto the top of the gradient and centrifuged for 1.5 h at 26,500 rpm in a Beckman SW41 rotor. Fractions were collected from the top of the gradient. Aliquots were resuspended in PBS and centrifuged for 1 h at 40,000 rpm in a Beckman Ti50.3 rotor. The resulting pellets were resuspended in RIPA buffer and resolved using SDS-PAGE, followed by either Coomassie blue staining or Western blot analysis.

Subtilisin Digestion Assay

Subtilisin digestion assays were performed essentially according to Ott et al (Ott et al., 1995). The purified virions were mock treated or treated with 1 mg/ml of subtilisin (Boehringer Mannheim) in digestion buffer (10 mM Tris-HCl, pH 8, 1 mM CaCl₂ and BSA) for 16 h at 37° C. Subtilisin was inactivated by phenylmethylsufonyl fluoride. Virions were then repelleted, resuspended in 2× loading buffer (120 mM Tris-HCl, pH6.8, 20% glycerol, 4% SDS, 200 mM DTT, 0.002% w/v Bromephenol blue) and subjected to SDS PAGE, followed by Western blot analysis, using anti-LysRS, anti-p24 and anti-gp120.

Expression and Purification of Recombinant Human Lysyl-tRNA Synthetase

His₆-tagged full length human LysRS was overexpressed in Escherichia coli, and purified as previously described (Shiba et al., 1997).

Results

LysRS is Incorporated Non-Randomly into HIV-1

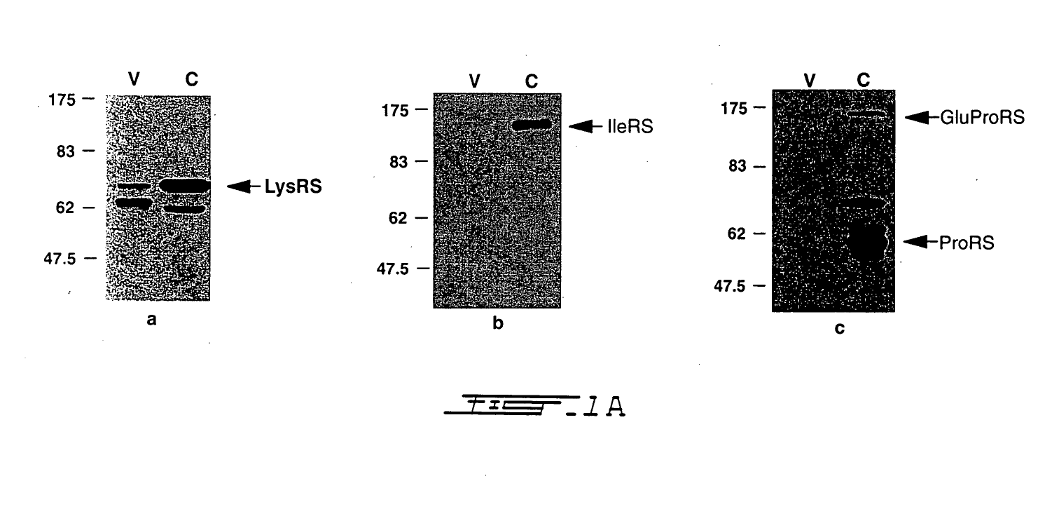

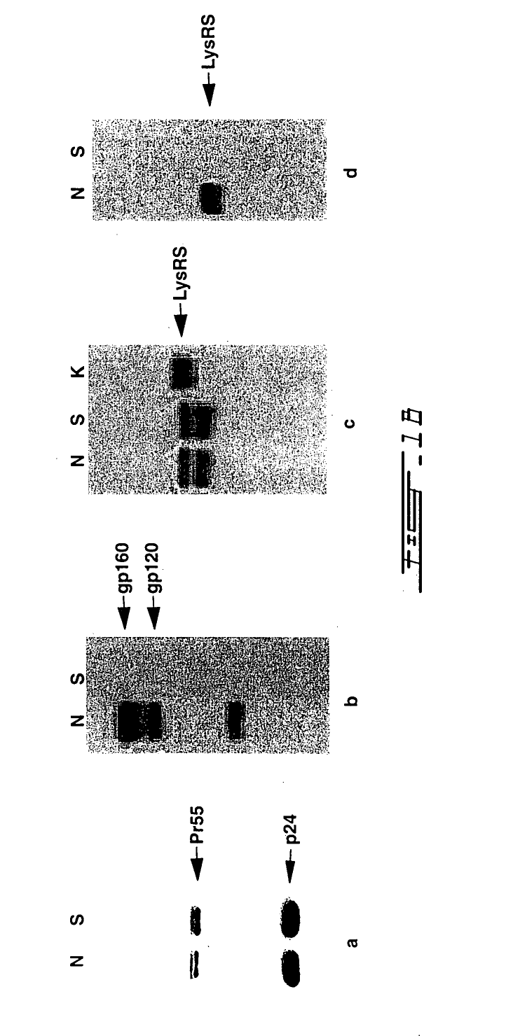

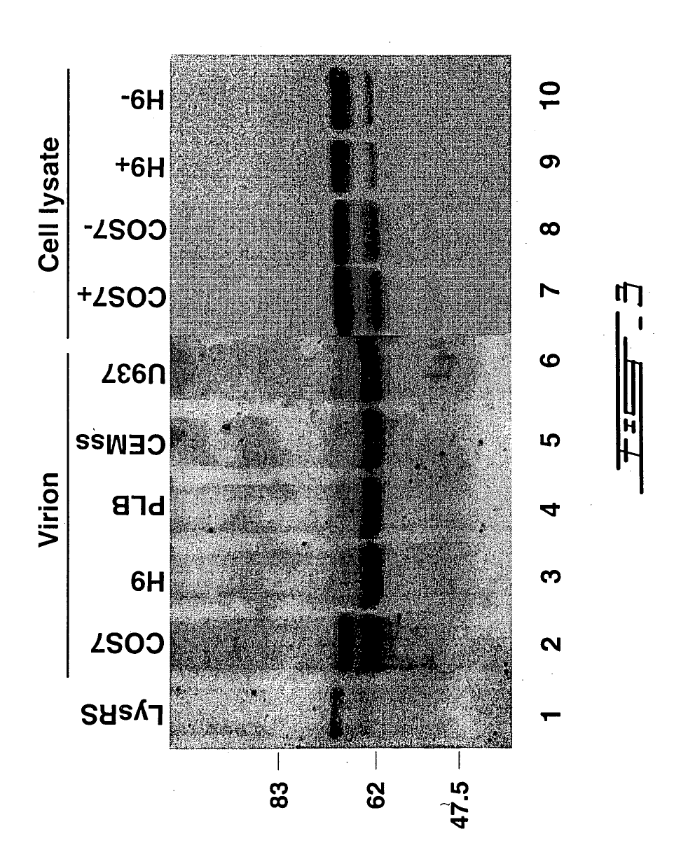

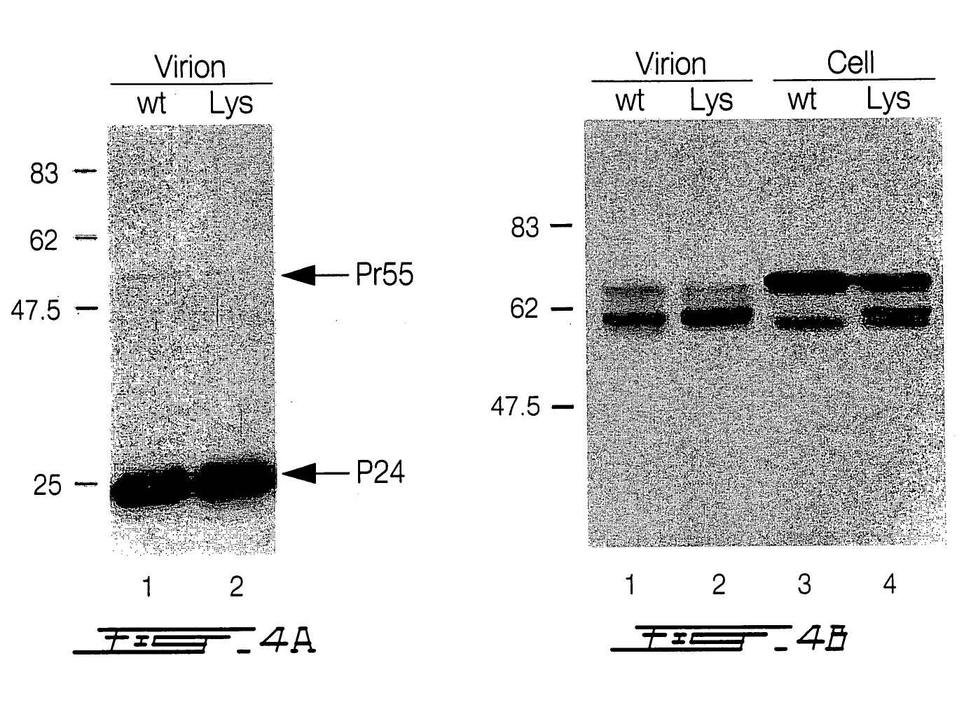

FIG. 1A shows Western blots of some aminoacyl-tRNA synthetases found in the cytoplasm of COS7 cells transfected with HIV-1 and in the viruses produced. Panel a represents a Western blot of either viral (V) or cytoplasmic (C) proteins probed with an antibody to human LysRS. In both the COS cell cytoplasm and in the viruses, LysRS species can be detected in three sizes. The apparent molecular weights (Mᵣ's) of these peptides, determined by SDS PAGE (FIGS. 1 and 3), are 70,000 for the large species, 63,000 for the intermediate species, and 62,000 for the small species. The large species predominate in the cytoplasm, while in the virus, both large and intermediate species are present. The sizes of the LysRS species determined by SDS PAGE are only approximate sizes since the calculated size of the human LysRS coded by a full length LysRS cDNA is 597 aa protein, with an Mᵣ of 68,034 (Shiba et al., 1997).

FIG. 1A also shows Western blots of cytoplasmic or viral protein probed with antibodies to human isoleucyl-tRNA synthetase (IleRS) (panel b) or human prolyl-tRNA synthetase (ProRS) (panel c). Human IleRS contains 1266 amino acid residues, with an Mᵣ of approximately 152,000 (Shiba et al., 1994). In all higher eukaryotes examined, ProRS is the C-terminal part of a fusion with GluRS (Cerini et al., 1991; Heacock et al., 1996), while the purified ProRS has an Mᵣ of approximately 60,000 (Ting et al., 1992). While these proteins are detected in the cytoplasm, they are not detected in the viruses, indicating that incorporation of LysRS into viruses is non-random.

The presence of LysRS within the virus is further substantiated by its resistance to digestion by the protease subtilisin (FIG. 1B). Intact viruses were either untreated (N) or treated with subtilisin (S) before viral lysis, and Western blots were probed with anti-p24 (panel a), anti-gp120 (panel b), and anti-LysRS (panel c). The results show that p24, Pr55^{gag}, and LysRS are resistant to proteolysis, while external proteins gp160 and gp120 are susceptible to proteolysis by subtilisin. This indicates that LysRS is present within the virus. Lane K contains purified, His₆-tagged human LysRS, which in panel c has not been exposed to protease. However, exposure of this purified protein to subtilisin does degrade it (panel d). The His₆-tagged human LysRS migrates more slowly than the large cytoplasmic LysRS species because of the N-terminal MRGSHHHHHHSSGWVD sequence appended to the full-length human LysRS used in these studies (Shiba et al., 1994).

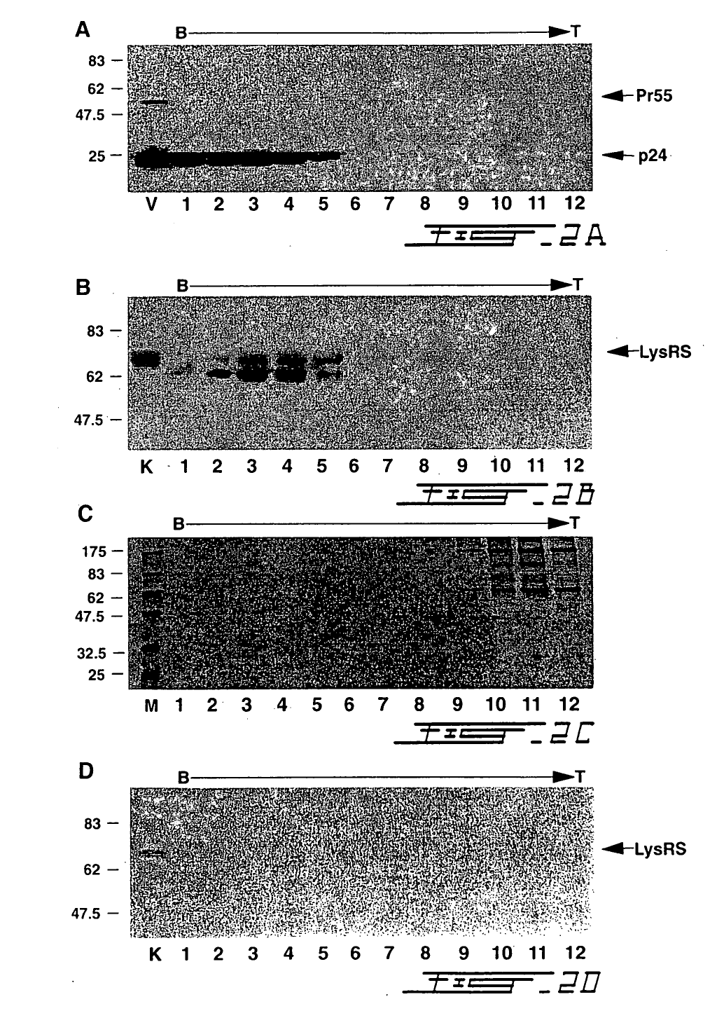

The virions studied in this work are purified by centrifugation through 15% sucrose to the surface of a 65% sucrose cushion. To further confirm that these viruses do not contain contaminating LysRS bound to their surface, viruses were also purified using velocity centrifugation through a 6-18% iodixanol gradient (Optiprep, Nycomed Pharma, Norway) instead of centrifugation through sucrose. Optiprep gradients have been shown to produce viruses more free from cytoplasmic contaminants than obtained using sucrose gradients (Dettenhofer et al., 1999). FIG. 2 shows Western blots of gradient fractions probed with anti-p24 (panel A) and anti-LysRS (panel B) following Optiprep gradient purification. We observe that LysRS comigrates with the viral Pr55^{gag} protein. Panel C shows the different gradient fractions stained with Coomassie Blue, and indicates that most residual cellular protein is found in fractions closer to the top of the gradient rather than where viral protein and LysRS migrate. Twenty times more viral lysate than used in panels A and B was used to visualize the proteins by Commassie Blue staining. Although the LysRS is detected in the same Optiprepl gradient fractions as p24, the LysRS/p24 ratio is much smaller in the heavier fractions 1 and 2 than in fractions 3-5. The bottom-most fractions could represent aggregates of broken virus no longer containing LysRS, or the anti-LysRS may have a lower sensitivity than anti-p24. In panel D, cell culture medium from non-transfected COS7 cells was resolved in the Optiprep gradient, and probing with anti-LysRS shows the absence of LysRS in the medium.

Sizes of LysRS Incorporated into HIV-1 Produced from Transfected COS7 Cells and Chronically-Infected Cell Lines

Although both large and intermediate size LysRS species are found in HIV-1 produced from COS7 cells, the intermediate size peptide is the major LysRS found in HIV-1 produced from chronically infected cell lines. This is shown in the Western blots probed with anti-LysRS in FIG. 3. In the cytoplasm of H9 cells, uninfected (lane 10) or chronically-infected with HIV-1 (lane 9), the major LysRS species is the large species, with a small amount of small species also present. Similar results are also found in the cytoplasm of PLB, CEMss and U937 cells (data not shown). On the other hand, in virions produced from these four chronically infected cell lines, the major LysRS species packaged is the intermediate size LysRS species.

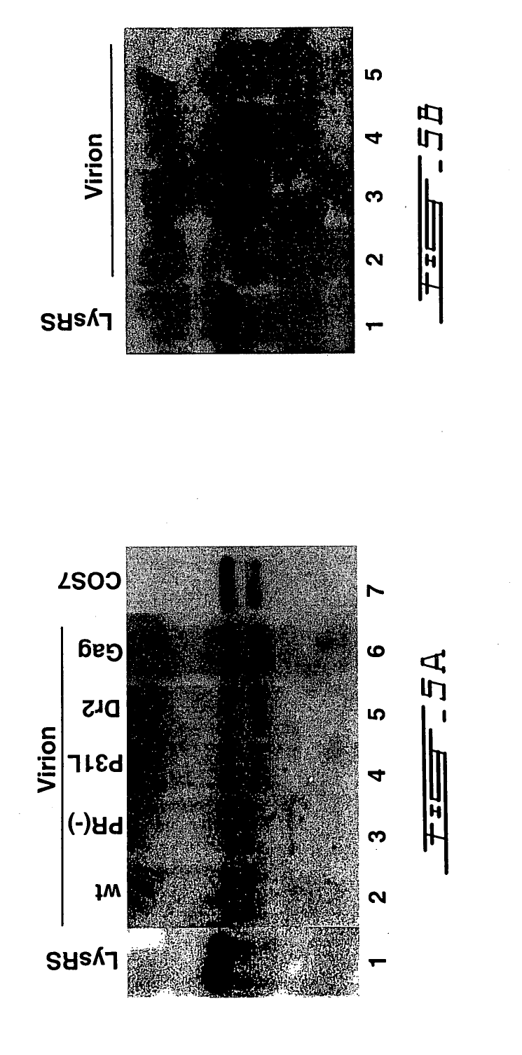

The ratio of intermediate to large LysRS species found in HIV(COS) can be influenced by the amount of tRNA^{Lys3} synthesized in the cell and packaged into the virion. It has already been shown that transfection of COS cells with a vector containing both HIV-1 proviral DNA and a tRNA^{Lys3} gene, results in an increase in tRNA^{Lys3} in the cytoplasm and in the virus (Huang et al., 1994). In FIG. 4, the effect of excess tRNA^{Lys3} on the level of LysRS in the cytoplasm and in the virus was analyzed. COS cells were transfected with either wild type (wt) HIV-1 proviral DNA or a plasmid containing both wt HIV-1 proviral DNA and a tRNA^{Lys3} gene. The amount of p24 present in each viral preparation was determined by western blot of viral protein probed with anti-p24 (see panel A). In panel B, viral protein containing equal amounts of p24 were blotted and probed with anti-LysRS (lanes 1 and 2). It can be seen that virions produced from cells with excess tRNA^{Lys3} also contain an excess of the intermediate species of LysRS. Indeed, densitometry analysis indicated that there was a 3-fold increase in the intermediate LysRS species as compared to that found in wild type viruses. The presence of the intermediate form of LysRS is also increased in the cytoplasm of these cells (see lanes 3 and 4 in panel B).

Taken together, such results show a positive correlation between the quantity of tRNA^{Lys3} and that of LysRS inside the virions.

Relationship Between LysRS and tRNA^{Lys} Incorporation in HIV-1

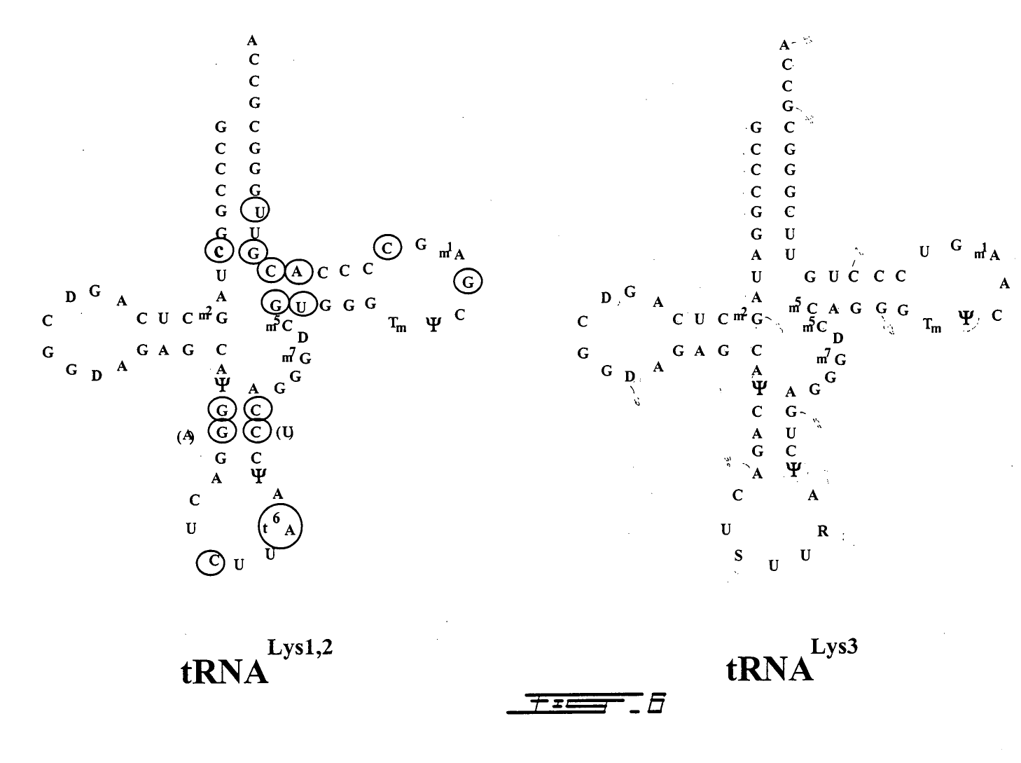

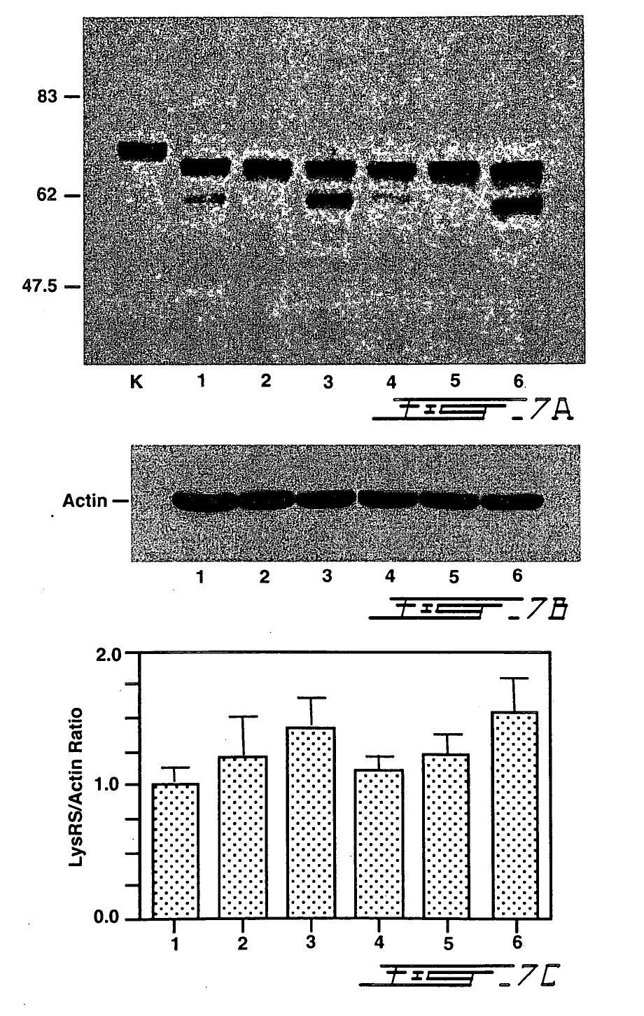

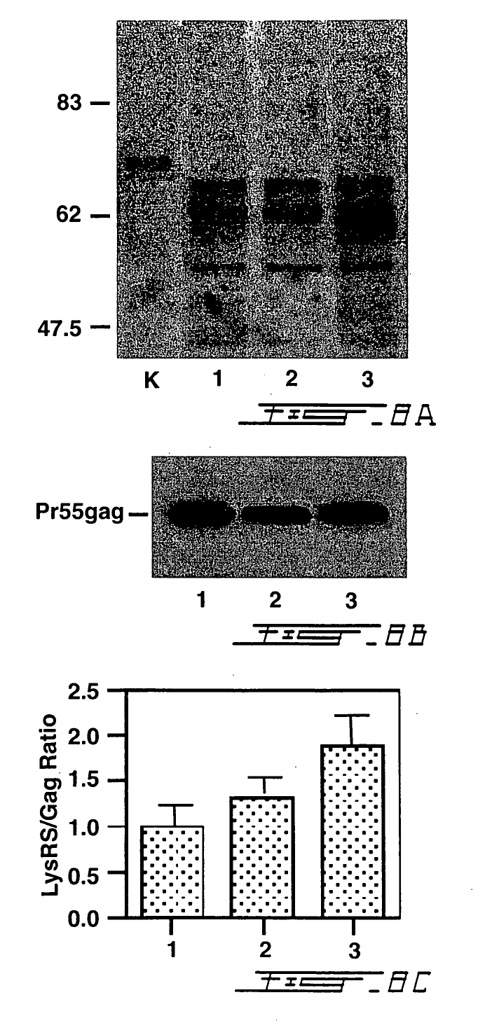

Mutant viruses previously shown to be deficient in tRNA^{Lys} incorporation (Huang et al., 1997; Mak et al., 1997) were produced by transfecting COS7 cells with wild type and mutant HIV-1 proviral DNA, and the incorporation of LysRS into the virions was analyzed by Western blots, as shown in FIG. 5. Lanes 1 and 7 show purified His₆-tagged-LysRS and LysRS found in COS7 cell cytoplasm, respectively. Lanes 2 and 3 represent protein from wild-type (wt) or protease-negative (PR(−)) viruses, respectively. Both viruses have been shown to selectively incorporate tRNA^{Lys} (Jiang et al., 1993; Khorchid et al., 2000), and lanes 2 and 3 show they both contain the large and intermediate size species of LysRS. Lanes 4-6 represent Western blots of protein from mutant viral-like particles (VLPs) P31L, Dr2, and Pr55^{gag}, none of which incorporate either Pr160^{gag-pol} or tRNA^{Lys} (Huang et al., 1997; Khorchid et al., 2000; Mak et al., 1994; Mak et al., 1997). P31L contains a substitution of P for L at position 31 in nucleocapsid protein (NCp7) in the basic amino acid sequence between the two Cys-His boxes. This mutation causes the rapid degradation of P160^{gag-pol} in the cytoplasm (Huang et al., 1997). Dr2 is a substitution mutation in the connection domain of RT, in which F₃₈₉ is replaced with F₃₈₉AG, and also causes the rapid degradation of Pr160^{gag-pol} in the cytoplasm (Mak et al., 1997). Lane 6 represents protein from Pr55^{gag} VLPs produced by transfecting COS cells with the vector pSVGAG-RRE, which codes only for Pr55^{gag} (Smith et al., 1993). These three different VLPs, which do not selectively package tRNA^{Lys}, do not contain the intermediate size LysRS species, but do contain the large and small species of LysRS. Thus, the incorporation of LysRS into viral particles appears dependent upon Pr55^{gag} protein, and is independent of tRNA^{Lys} or Pr160^{gag-pol} incorporation. However, the presence of intermediate size LysRS in viruses appears to be directly correlated with the packaging of tRNA^{Lys} and Pr160^{gag-pol}. We have previously reported that selective packaging of tRNA^{Lys} can be partially rescued in the P31L VLP by cotransfection of COS cells with P31L proviral DNA and DNA coding for wild type Pr160^{gag-pol}, but not with DNA coding for wild-type Pr55^{gag} (Huang et al., 1997). The effect of the rescue of tRNA^{Lys} packaging upon LysRS incorporation was investigated next. FIG. 5B shows a Western blot probed with anti-LysRS, containing purified His₆-tagged LysRS (lane 1), and protein from protease-negative HIV-1, which packages tRNA^{Lys} and which shows the large and intermediate size LysRS species (lane 2). Lane 3 contains protein from the P31L mutant, which does not package tRNA^{Lys}, Pr160^{gag-pol}, or the intermediate size LysRS. Cotransfection with pSVFS5TprotD25G, which codes for wild type Pr160^{gag-pol}, and which partially rescues tRNA^{Lys} packaging, also results in a small amount of intermediate size LysRS incorporation (lane 4). In contrast, cotransfection with pSVGAG-RRE-R, which codes for wild type Pr55^{gag}, and which does not rescue tRNA^{Lys} packaging, also does not result in the incorporation of intermediate size LysRS (lane 5).

Discussion

In this work, we have provided evidence for the incorporation of human LysRS into HIV-1. This evidence included detection of LysRS in virions purified by centrifugation using either sucrose or Optiprep gradients. Two other human aminoacyl-tRNA synthetases, ProRS and IleRS, were not detected in virions, though they were readily detected in the cytoplasm of HIV-1-transfected cells. While purified LysRS was susceptible to degradation by the protease subtilisin, LysRS detected in viruses was resistant to subtilisin digestion under reaction conditions in which external envelope protein gp120 was degraded.

We detect LysRS in three sizes, with apparent molecular weights on SDS gels of 70,000 (large species), 63000 (intermediate species), and 62,000 (small species). The results in FIG. 5 indicate that Pr55^{gag} alone among the viral proteins is sufficient for incorporating LysRS. The Gag VLPs do not incorporate either tRNA^{Lys} or Pr160^{gag-pol}, and the intermediate LysRS is replaced with the small species. The three types of Pr55^{gag} VLPs (FIG. 5A, lanes 4-6) do not incorporate either tRNA^{Lys} or Pr160^{gag-pol}. The viral-like particles which contain only Pr55^{gag} (FIG. 5A, lane 6) are produced by cotransfecting cells with pSVGAG-RRE-R and pCMV-REV. The HIV-1 proviral DNA in the former plasmid not only lacks viral sequences downstream of Gag (except for the RRE), but an SV40 late promoter region has replaced all viral sequences upstream of nucleotide 679 in the viral DNA. The viral-like particles produced are defective in incorporating the truncated genomic RNA as well as tRNA^{Lys} and Pr160^{gag-pol} (Mak et al., 1994; Smith et al., 1990; Smith et al., 1993). Pr55^{gag} may interact with a cytoplasmic tRNA^{Lys}/LysRS complex and destabilize it, thereby releasing the tRNA^{Lys} and resulting in the incorporation of LysRS alone into the Gag VLP. The additional presence of Pr160^{gag-pol} may serve to stabilize the Pr55^{gag}/tRNA^{Lys}/LysRS ternary complex since Pr160^{gag-pol} interacts with both tRNA^{Lys} (Khorchid et al., 2000) and Pr55^{gag} (Park et al., 1992; Smith et al., 1993).

Destabilization of the LysRS/tRNA^{Lys} complex by the large number of Pr55^{gag} molecules in the cell might be expected to inhibit translation. There are a number of possible reasons why this does not happen. Most Pr55^{gag} molecules may not bind LysRS, either because Pr55^{gag} molecules without Pr160^{gag-pol} have a weaker affinity for LysRS, or because Pr55^{gag} only interacts with LysRS as a multimeric Pr55^{gag} complex. Additionally, the destabilization of tRNA^{Lys}/LysRS may release free non-acylated tRNA^{Lys}, a molecule which has been shown in yeast to induce the synthesis of more LysRS (Lanker et al., 1992), which could help maintain the cytoplasmic concentrations of tRNA^{Lys}/LysRS and lysine-tRNA^{Lys} required for translation.

We do not yet know if Pr55^{gag} interacts directly with LysRS. Since the plasmid coding for the Pr55^{gag} protein, pSVGAG-RRE-R, codes only for this protein, (Smith et al., 1990), Vpr, a viral protein which was shown to interact with human LysRS both in vitro and in the yeast two hybrid system (Stark et al., 1998), is not needed for the incorporation of LysRS into the Pr55^{gag} particles. We have also previously shown that tRNA^{Lys} is selectively incorporated into HIV-1 missing Vpr (Khorchid et al., 2000). On the other hand, Pr55^{gag} might interact indirectly with LysRS via another cellular tRNA-binding protein, such as elongation factor 1-alpha, which has been shown to interact with Pr55^{gag} and to be incorporated into HIV-1 during assembly (Cimarelli et al., 1999).

The dominant LysRS form in viruses produced from the human cell lines is the intermediate form (FIG. 3). Since truncation of LysRS to the small species also occurs in Gag VLPs, processing does not depend upon the presence of either Pr160^{gag-pol} or tRNA^{Lys}, but may be limited by them to produce the intermediate species. The predominance of large LysRS in the cytoplasm and intermediate LySRS in the viruses (particularly in viruses produced from human cell lines) suggests that the intermediate and small LysRS species may be generated by proteolysis of the large species, a phenomenon observed during the in vitro proteolytic cleavage of the N terminal regions of dimeric yeast (Ciracoglu et al., 1985) or sheep (Cirakoglu et al., 1985) LysRS to truncated homodimers. The detection of LysRS heterodimers in sheep has also been reported (Cirakoglu et al., 1985). However, if a protease is involved, it is not a viral protease since processing of LysRS occurs in both Gag VLPs and in protease-negative virions. A recent report does indicate that the human cytoplasmic and mitochondrial LysRSs are generated by alternative splicing of the same primary RNA transcript (Tolkunova et al., 2000). The mitochondrial LysRS contains extra amino acid sequences used for mitochondrial targeting in the N-terminal region, and because it is larger than the cytoplasmic LysRS, it is unlikely to be represented by the intermediate and small species observed in the present studies. Alternate RNA splicing has also been reported for generating human cytoplasmic cysteinyl-tRNA synthetase (Kim et al., 2000).

Very little processed LysRS is detected in the cytoplasm of chronically-infected cell lines (FIG. 3), and this is the small species. These data presented herein therefore appear to support the possibility that the processing of the large LysRS species to the intermediate species occurs during or after viral release from the cell. We cannot exclude the possibilities that either non-detectable amounts of intermediate LysRS in the cytoplasm are selectively packaged into the virus, or that the scarcity of the intermediate LysRS species in the cytoplasm is due to the fact that it is selectively packaged into the virus. The presence of both large and intermediate species of LysRS in HIV-1 produced from COS7 cells does indicate that the large species is capable of being packaged into the virion, however. While the ratio of intermediate to large LysRS species varies from one preparation of HIV (COS) to the next (for example, compare FIG. 1C with FIG. 1A or 4B), it is usually greater than 1 and increases when tRNA^{Lys3} packaging increases (FIG. 4B).

It has been shown that removal of N-terminal sequence from yeast AspRS weakens binding of the enzyme to the tRNA^{Asp}, as shown by an increase in both the Kd for tRNA binding and K_{M} of the aminoacylation reaction of approximately 2 orders of magnitude (Frugier et al., 2000). On the other hand, human LysRS missing the N-terminal 65 amino acids did not display significantly reduced in vitro aminoacylation kinetics (Shiba et al., 1994), implying a similar tRNA^{Lys} binding affinity as for wild type LysRS. Of note, the removal of the N-terminal extension of human LysRS, absent in prokaryotic enzymes, was shown to be dispensable for its in vitro aminoacylation activity and for the in vivo cross-species complementation from human to E. coli (Shiba et al., 1997). Reduced affinity of the intermediate LysRS for tRNA^{Lys} might therefore be due to other LysRS sequences missing, or to a cellular environment different from that tested in vitro.

### EXAMPLE 3

### Regulation of tRNA^{Lys} Incorporation into HIV-1 by Lysyl tRNA Synthetase

We have shown that during HIV-1 assembly in COS7 cells transfected with HIV-1 proviral DNA, lysyl tRNA synthetase (LysRS) and the major tRNA^{Lys} isoacceptors, tRNA^{Lys1,2} and tRNA^{Lys3}, are selectively packaged into the viruses. Pr55^{gag} alone is sufficient for packaging LysRS into Pr55^{gag} particles, but the additional presence of Pr160^{gag-pol} is required for tRNA^{Lys} incorporation as well. Since Pr160^{gag-pol} interacts with both Pr55^{gag} (Park et al., 1992; Smith et al., 1990) and tRNA^{Lys} (Khorchid et al., 2000; Mak et al., 1994), its presence may stabilize the Pr55^{gag}/LysRS/tRNA^{Lys} complex. It is not known if Pr55^{gag} interacts directly with LysRS or through another cellular tRNA-binding protein, such as elongation factor 1-alpha (EF1∀). EF1∀ has been shown to interact directly with Pr55^{gag} and to be incorporated into HIV-1 during assembly (Cimarelli et al., 1999) On the other hand, Vpr, a viral protein which was shown to interact with human LysRS in vitro and in the yeast two hybrid system (Stark et al., 1998), is not needed for the incorporation of LysRS into the Pr55^{gag} particles, since plasmids used to produce Pr55^{gag} viral-like particles which package LysRS did not code for Vpr (Example 2), and tRNA^{Lys} is also selectively incorporated into Vpr-negative HIV-1 (Mak et al., 1994). Whether Vpr plays another role, such as in facilitating tRNA^{Lys3} genomic placement or deacylating tRNA^{Lys3}, is not yet known.

In the cytoplasm of uninfected or infected cells, SDS PAGE indicates that there exists both an abundant LysRS species with an apparent molecular weight of approximately 68,000 (large species), and a smaller less abundant species with an approximate molecular weight of 62,000 (small species). In HIV-1 produced from a number of cell lines, the predominant LysRS species has an intermediate molecular weight of 63,000 (Example 2). In HIV-1 produced from COS7 cells, both large and intermediate LysRS species are present, usually in similar amounts. The intermediate species is always present in viruses incorporating tRNA^{Lys}. The production of virus-like particles (VLPs) composed only of Pr55^{gag} is sufficient for incorporation of LysRS. However, tRNA^{Lys} is not selectively packaged into these particles, and only the large and intermediate LysRS species are present in the viruses. Since the intermediate species is present in protease-negative viruses, and the small species in Pr55^{gag} VLPs (Example 2), the intermediate and small species could not be generated by a viral protease. The precise nature of the modification of these LysRS species is not yet known, and appears to be due to a cellular protease (Cirakoglu et al., 1985). Alternatively, it could be due to alternative splicing of the same primary RNA transcript (Tolkunova et al., 2000). LysRS truncation could result in weakening the interaction between LysRS and tRNA^{Lys} (Frugier et al., 2000) which might facilitate either tRNA^{Lys} interaction with viral proteins during packaging or annealing to the viral genomic RNA.

Herein, it is shown that tRNA^{Lys} packaging is limited by the level of LysRS, since the overproduction of LysRS from a cotransfected plasmid encoding LysRS results in up to a 2 fold increase in the incorporation of both tRNA^{Lys} isoacceptors into the viruses. Overproduction of LysRS also results in an increase in both LysRS packaging into HIV-1 and in the cytoplasmic concentrations of both tRNA^{Lys} isoacceptors. However, increased cytoplasmic concentrations of tRNA^{Lys} are not the prime cause of increased tRNA^{Lys} incorporation into viruses. Overproduction of a mutant LysRS lacking the N terminal 65 amino acids also results in increases in both LysRS viral packaging and tRNA^{Lys} concentrations in the cytoplasm, but no increase in tRNA^{Lys} viral packaging is observed. This probably reflects the weaker affinity the mutant LysRS has for tRNA^{Lys}, as demonstrated by electrophoretic band shift assays of in vitro tRNA^{Lys3}/LysRS binding. Wild type LysRS can migrate to the nucleus, but since the N-terminal mutant LysRS has lost this ability, increased tRNA^{Lys} gene expression is not due to a direct stimulation of transcription or nuclear export by LysRS.

Materials and Methods

Plasmid Construction

SVC21.BH10 P- is a simian virus 40-based vector that contains full-length wild-type HIV-1 proviral DNA containing an inactive viral protease (D25G), and obtained from E. Cohen, University of Montreal. pM368 contained cDNA encoding full length (1-597 amino acids) human LysRS, was obtained from Shiba et al., 1997. The cDNA was PCR-amplified, and digested with EcoR1 and Xho1, whose sites were placed in each of the PCR primers. To produce an N-terminal truncated LysRS encoding amino acids 66-597, the sense primer was complementary to a downstream sequence. For expression in COS7 cells, the PCR DNA fragments were cloned into either pcDNA 3.1 (Invitrogen) to obtain pLysRS.F and pLysRS.T, expressing full length or N-terminal truncated LysRS, respectively, or into pcDNA3.1/V5-HisA, which adds C-terminal tags V5 and His₆ to the wild type (LysRS.CF) and mutant (LysRS.CT) LysRS species. To purify the wild type and mutant LysRS, the PCR DNA fragments were cloned into the bacterial expression vector pET-21b(+) (Clonetech), which expresses the proteins with a C-terminal His₆ tag. His₆-tagged full length and truncated human LysRS was overexpressed in Escherichia coli, and purified as previously described (Shiba et al., 1997).

Cell Culture and Fractionation

COS7 cells were maintained in Dulbecco modified Eagle medium with 10% fetal bovine serum and antibiotic. For cell fractionation, cells were resuspended in lysis buffer (PBS with 0.1% Nonidet P-40, 0.1% Triton X-100, and protease inhibitor coctail tablets (Roche)), and incubated on ice for 10 minutes. Nuclei were pelleted by centrifugation at 1000×g for 10 minutes at 4EC, and the supernatant was collected as the cytoplasmic fraction. Nuclear extracts were prepared by lysing nuclei in RIPA buffer. Western blot analysis of the total cell lysate, postnuclear supernatant and nuclear extracts were performed as described below, using anti V5 (Invitrogen), anti-tubulin (Santa Cruz Biotechnology) and anti-YYI (Santa Cruz Biotechnology). Anti-V5 was used to detect LysRS.CF and LysRS.CT, wild type and mutant LysRS which contain a C-terminal 14 amino acid V5 epitope.

Production of Wild-Type and Mutant HIV-1 Virus

Transfection of COS7 cells with the above plasmids by the calcium phosphate method was as previously described (Mak et al., 1994). Viruses were isolated from COS7 cell culture medium 63 h posttransfection, or from the cell culture medium of infected cell lines. The virus-containing medium was first centrifuged in a Beckman GS-6R rotor at 3,000 rpm for 30 minutes and the supernatant was then filtered through a 0.2 μm filter. The viruses in the filtrate were then pelleted by centrifugation in a Beckman Ti45 rotor at 35,000 rpm for 1 h. The viral pellet was then purified by centrifugation with a Beckman SW41 rotor at 26,500 rpm for 1 h through 15% sucrose onto a 65% sucrose cushion.

RNA Isolation and Analysis

Total cellular or viral RNA was extracted from cell or viral pellets by the guanidinium isothiocyanate procedure (Chomczynski et al., 1987), and dissolved in 5 mM Tris buffer, pH 7.5. Hybridization to dot blots of cellular or viral RNA were hybridized with DNA probes complementary to tRNA^{Lys3} and tRNA^{Lys1,2} (Jiang et al., 1993), genomic RNA (Cen et al., 1999), and 3-actin mRNA (DNA probe from Ambion). 2D PAGE of ³²pCp-3′ end labeled viral RNA was carried out as previously described (Jiang et al., 1993).

Western Blotting

Sucrose-gradient-purified virions were resuspended in 1× radioprecipitation assay buffer (RIPA buffer: 10 mM Tris, pH 7.4, 100 mM NaCl, 1% deoxycholate, 0.1% sodium dodecyl sulfate (SDS), 1% Nonidet P-40, protease inhibitor cocktail tablets (Boehringer Mannheim)). Western blot analysis was performed using either 300 μg of cellular protein or 10 μg of viral protein, as determined by the Bradford assay (Bradford et al., 1976). The cellular and viral lysates were resolved by SDS polyacrylamide gel electrophoresis (SDS-PAGE), followed by blotting onto nitrocellulose membranes (Gelman Sciences). Detection of protein on the Western blot utilized monoclonal antibodies or antisera specifically reactive with viral p24 (mouse antibody, Intracel), 3-actin (Sigma Aldrich), and human LysRS (rabbit antibody, obtained from K. Shiba (Shiba et al., 1997)). Western blots were analyzed by enhanced chemiluminescence (ECL kit, Amersham Life Sciences) using goat anti-mouse or donkey anti-rabbit (Amersham Life Sciences) as a secondary antibody. The sizes of the detected protein bands were estimated using pre-stained high molecular mass protein markers (GIBCO/BRL).

Electrophoretic Band Shift Assay

tRNA^{Lys} was purified from human placenta as previously described (Jiang et al., 1993), and labeled with the 3′-³²pCp end-labeling technique as previously described (Bruce et al., 1978). In 20:1 binding buffer (20 mM Tris-HCl, pH 7.4, 75 mM KCl, 10 mM MgCl₂, and 5% glycerol) 5 nM labeled tRNA^{Lys} was incubated with different concentrations of LysRS (0.06 uM, 0.3 uM, or 1.5.uM) for 15 minutes on ice, and then analyzed by 1 D-PAGE (native 6% gels in 1×TBE at 4° C.).

Results

LysRS Overexpression: Effect upon Cytoplasmic and Viral Concentrations of LysRS and tRNA^{Lys} Isoacceptors

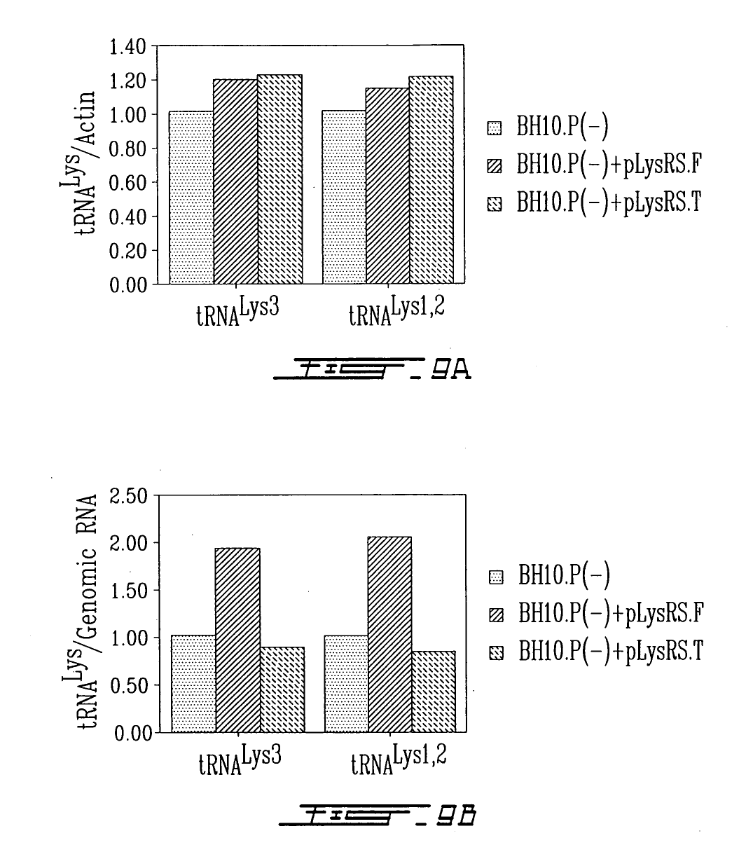

COS7 cells were transfected with plasmids coding for either full length LysRS (LysRS.F) or a truncated LysRS, in which the first N-terminal 65 amino acids have been deleted (LysRS.T). FIG. 7 shows western blots of cell lysates, probed with either anti-LysRS (panel A) or anti-actin (B). The bands were quantitated by phosphorimaging, and the LysRS/actin ratios are shown in panel C, normalized to the LysRS/actin in non-transfected COS7 cells. LysRS.T is expressed somewhat better than LysRS.F, as shown by the higher LysRS/actin ratios. These ratios are similar in cells without viruses (lanes 1-3) and in cells producing viruses (lanes 4-6).

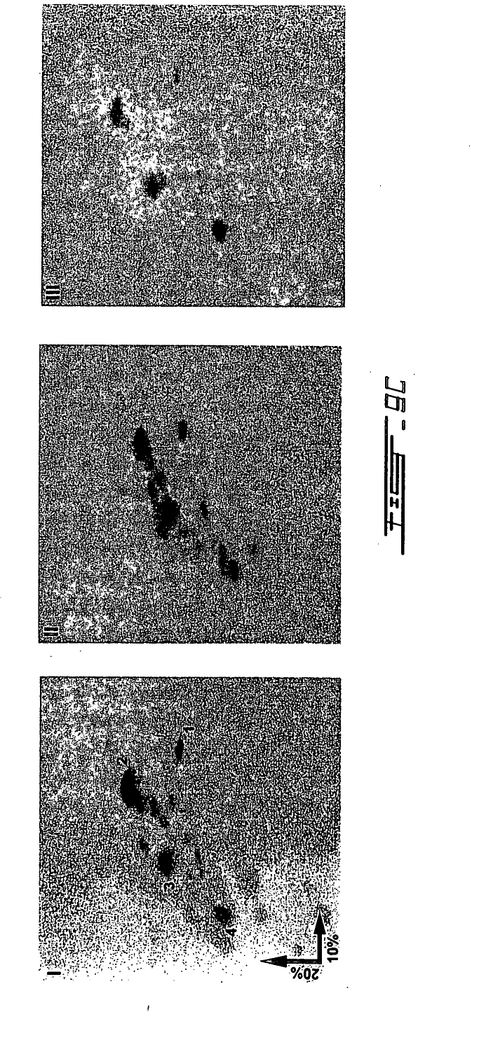

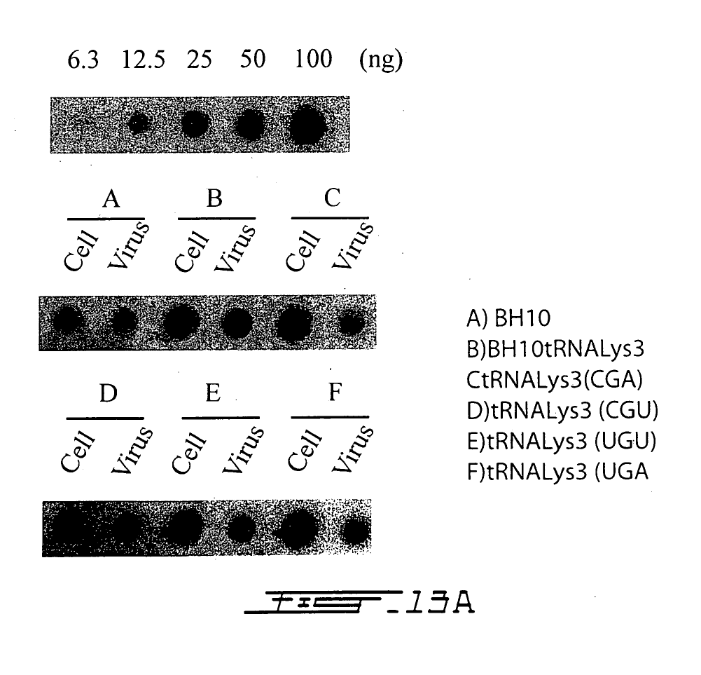

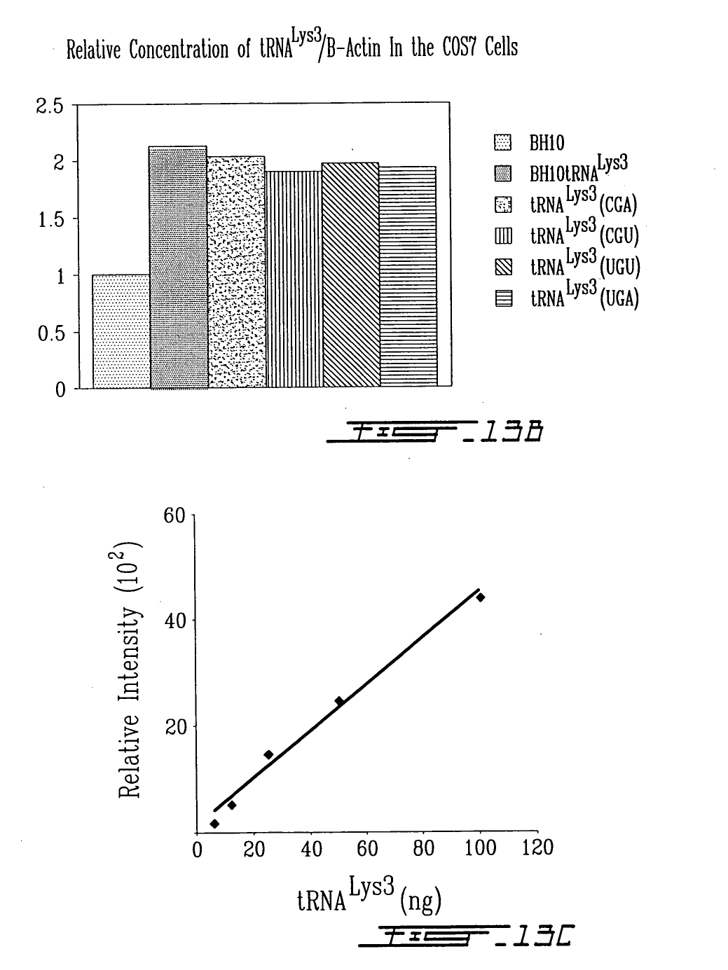

As shown in FIG. 8, the overexpression of LysRS in cells cotransfected with a plasmid (BH10P-) containing protease-negative HIV-1 proviral DNA results in increased packaging of LysRS in the viruses produced. FIG. 8A shows western blots of viral lysates probed with either anti-LysRS or anti-CA. As previously reported (Example 2), LysRS in virions produced from COS7 cells contains both the full length (large) LysRS, and the intermediate species. The bands were quantitated by phosphorimaging, and the LysRS/Gag ratios are shown in panel C, normalized to the Lys/Gag ratio for cells transfected with BH 10P- only (lane 1). It can be seen that more LysRS.T is incorporated into virions than LysRS.F, which may reflect the higher amount of LysRS.T present in the cytoplasm. This reduced overexpression of LysRS.F compared to LysRS.T may be due the ability of LysRS.F to feedback-inhibit its own synthesis by returning to the nucleus, something LysRS.T cannot do (See below and FIG. 11).

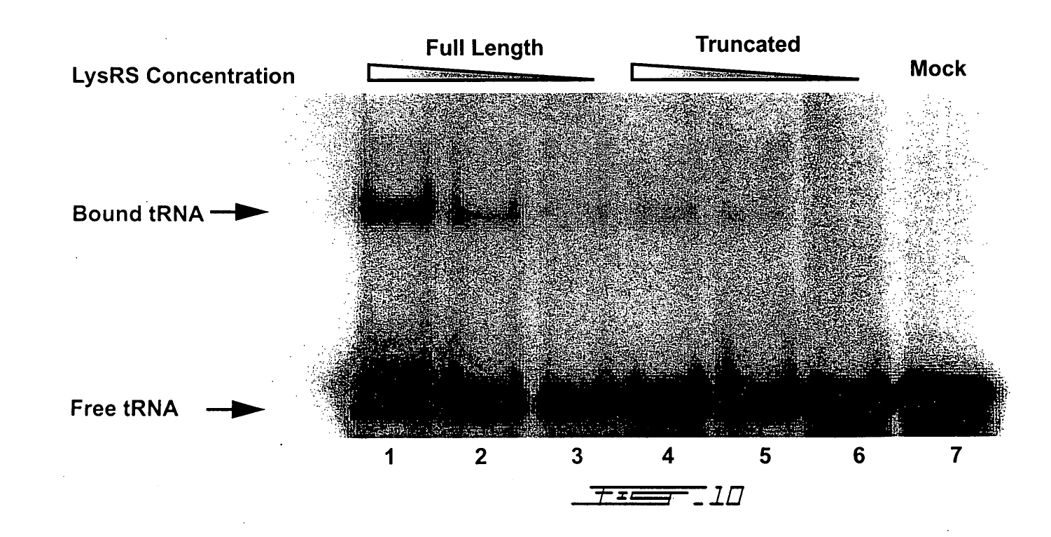

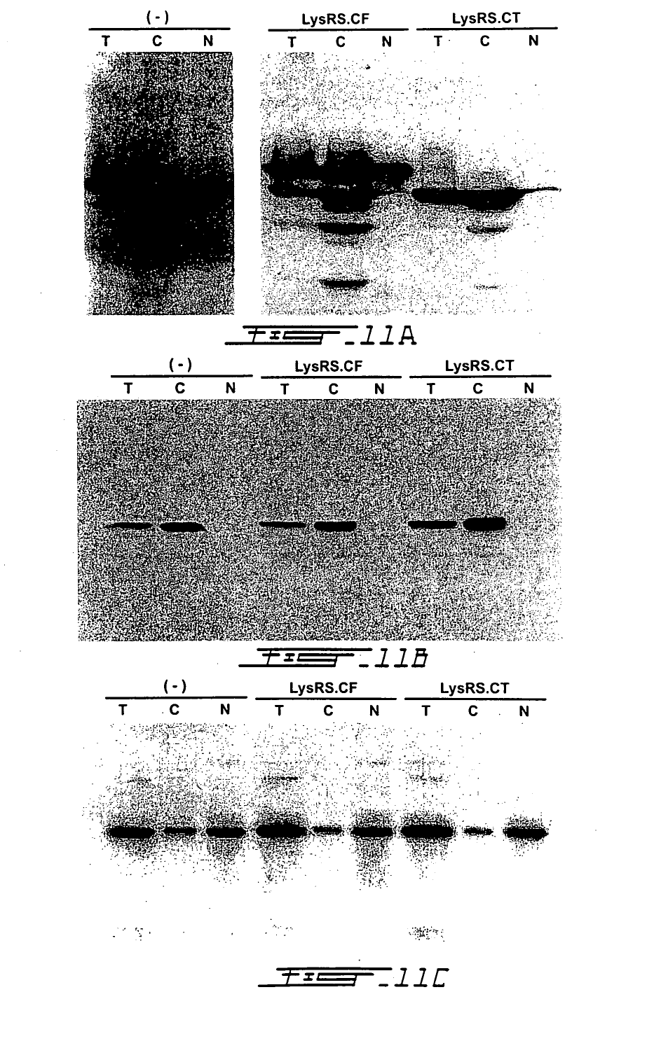

FIG. 9 shows the effect of LysRS overexpression upon tRNA^{Lys} concentrations in the cytoplasm of HIV-1-transfected-COS7 cells and in the virions produced from these cells. Dot blot, hybridization was used to determine the tRNA^{Lys3}/actin mRNA and tRNA^{Lys1,2}/actin mRNA ratios in total cytoplasmic RNA, using hybridization probes specific for these RNAs (Jiang et al. 1993). The results, quantitated by phosphorimaging, are shown in FIG. 9A. Small increases in the cytoplasmic concentrations of the major tRNA^{Lys} isoacceptors are seen using either LysRS.F or LysRS.T. In the experiments represented in panels B, dot blot hybridization of total RNA isolated from the virus produced in these cells was used to measure the tRNA^{Lys3}/genomic RNA and the tRNA^{Lys1,2}/genomic RNA ratios. The results, quantitated by phosphorimaging, are shown in panel B. It is quite clear that only the overexpression of LysRS.F results in an increase in the incorporation of tRNA^{Lys} isoacceptors into the viruses.

This result can also be seen in panel C, which shows the resolution of viral tRNA^{Lys} isoacceptors by 2D-PAGE. Total viral RNA samples containing equal amounts of genomic RNA were end-labeled with ³²pCp, and the low molecular-weight RNA, which is the only RNA able to enter the gel, was resolved by 2D-PAGE. The position of the tRNA^{Lys} isoacceptors is as previously determined (Jiang et al., 1993), and while the ratio of these isoacceptors to each other in the viruses does not change, it is clear that on a genomic RNA basis, the tRNAs in virions produced from cells overexpressing LysRS.F show the strongest signal, thereby supporting the conclusions derived from the dot blot hybridiation data in panel B, ie, overexpression of LysRS.T does not induce greater packaging of tRNA^{Lys} isoacceptors into the viruses. The slowest moving tRNA species, “4”, is not a tRNA^{Lys} isoacceptor, and has been tentatively identified as tRNA^{Asn} (data not shown), a tRNA species previously reported to be packaged into HIV-1 (Zhang et al., 1996). The spot 4:tRNA^{Lys} ratio appears to decrease upon expression of excess LysRS.F, but increases upon expression of LysRS.T.

LysRS.T Binds more Weakly to tRNA^{Lys3} In Vitro than LysRS.F

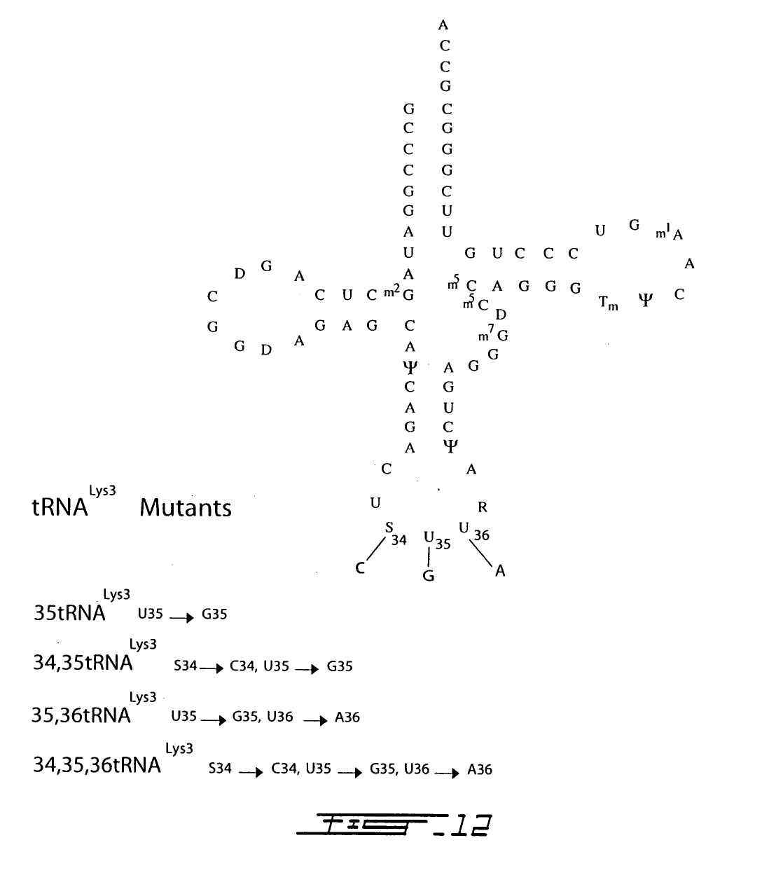

Overexpression of LysRS.T results in similar increases in both the cytoplasmic concentrations of tRNA^{Lys} and in viral incorporation of LysRS, yet, unlike LysRS.F, does not result in greater tRNA^{Lys} packaging into the virion. One explanation may be that LysRS.T cannot bind as well to tRNA^{Lys} isoacceptors as LysRS.F, and we have investigated this. N-terminal, His-tagged human LysRS, wild type or mutant, was purified by Ni++ chromatography (Shiba et al., 1997), and human tRNA^{Lys3} was purified from human placenta, as previously described (Jiang et al., 1993). The ability of LysRS to bind radioactive tRNA^{Lys3} in vitro was determined using an electrophoretic band shift assay, and the results are shown in FIG. 10. Human tRNA^{Lys3} was 3-end labeled with ³²pCp (Bruce et al., 1978), and incubated with increasing amounts of purified LysRS.T or LysRS.F. The resulting complexes were resolved on 1D-PAGE, and FIG. 10 indicates that LysRS.F has a greater ability to form complexes with the labeled tRNA^{Lys3} than does LysRS.T.

LysRS.T has lost the Ability to Migrate to the Nucleus

Because overexpression of either LysRS.F or LysRS.T results in an increase in the cytoplasmic concentrations of tRNA^{Lys} isoacceptors, we investigated the possibility of a direct derepression of tRNA^{Lys} genes by LysRS as a result of LysRS migrating into the nucleus. COS7 cells were transfected with plasmids coding for either LysRS.CF or LysRS.CT, where the “C” indicates that the LysRS has been C-terminally tagged with the 14 amino acid V5 epitope. Cells were lysed in 0.1% NP-40, and western blots were used to examine either the total lysate (T), or cell lysate fractionated by low speed centrifugation, into nuclear (N) and cytoplasm (C) compartments. FIG. 11A shows the distribution of wild type and mutant LysRS in the cell. The first 3 lanes detect endogenous LysRS in non-transfected cells, using anti-LysRS, and show that while both the full length and smaller LysRS appear in the total lysate and in the cytoplasm, as previously described (Example 2), only the full length LysRS can be seen in the nuclear fraction. The next 6 lanes use anti-V5 to detect exogenous full length (LysRS.CF) and experimentally truncated LysRS (LysRS.CT) in the different cell fractions. The expression of LysRS.CF in the cell does result in the generation of some smaller peptides in the cytoplasm, but clearly only the full length LysRS.CF is seen in the nucleus. Since the smaller fragments must contain the C-terminal tag V5 to be detected by anti-V5, the smaller fragments may have resulted from N-termnal deletions. In fact, as shown in the last three lanes, experimental deletion of the N-terminal 65 amino acids (LysRS.CT) results in the inability of this truncated LysRS to migrate to the nucleus. Panels B and C represent controls for the purity of the nuclear and cytoplasmic preparations, ie, the known cytoplasmic protein, alpha tubulin, is not detected in the nuclear fraction (panel B), while the nuclear transcription factor, YYI, is primarily found in the nucleus (panel C).

Discussion

Increasing the cytoplasmic concentration of wild type LysRS in COS7 cells by transfecting cells with LysRS.F results in an approximately 20% increase in the cytoplasmic concentration of tRNA^{Lys} and an approximately 2 fold increase in the incorporation of tRNA^{Lys3} and tRNA^{Lys1,2} into virions. This observed increase in viral incorporation of all major tRNA^{Lys} isoacceptors is in contrast to results previously obtained when overexpressing a particular tRNA^{Lys} isoacceptor. For example, transfection of COS7 cells with a plasmid coding for both HIV-1 proviral DNA and a tRNA^{Lys3} gene results in virions with an increased concentration of tRNA^{Lys3}, and a decreased concentration of tRNA^{Lys1,2}, indicating that some tRNA^{Lys} packaging factor has been saturated (Huang et al., 1994). Based on the results presented herein, it is strongly suggested that this factor is LysRS, since increases in cytoplasmic LysRS result in the increased incorporation of all the major tRNA^{Lys} isoacceptors.

The increase in tRNA^{Lys} packaging is not directly due to the increases in cytoplasmic tRNA^{Lys} concentrations, since overexpression of LysRS.T also results in increases in cytoplasmic tRNA^{Lys} concentrations, but no increase in viral incorporation of tRNA^{Lys} is observed. This is probably because LysRS.T does not bind to tRNA^{Lys} as well as LysRS.F (FIG. 10). It has been shown that removal of N-terminal sequence from yeast AspRS weakens the in vitro binding of the enzyme to the tRNA^{Asp}, as shown by an increase in both the Kd for tRNA binding and K_{M} of the aminoacylation reaction of approximately 2 orders of magnitude (Frugier et al., 2000). LysRS and AspRS are both class IIB synthetases, i.e., they are structurally similar. However, it has been reported that human LysRS missing the N-terminal 65 amino acids did not display significantly reduced in vitro aminoacylation kinetics using an in vitro synthesized tRNA^{Lys3} transcript (Shiba et al., 1997). This discrepency with our band shift observations might be due either to differences in interaction using natural tRNA^{Lys3} vs an unmodified tRNA^{Lys3} transcript, or to the much higher concentrations of tRNA^{Lys3} used in the in vitro aminoacylation reaction (50-800 fold higher than used in the band shift experiments reported here), which might mask reduced affinities.

The increase in cytoplasmic tRNA^{Lys} caused by overexpression of wild type or mutant LysRS could be due to several factors, including increased tRNA^{Lys} expression (ie, increased transcription or nuclear export) and/or increased tRNA^{Lys} stability. In yeast, uncharged tRNA^{Lys} acts thru a signal transduction pathway to activate the synthesis of more LysRS through increased transcription of the LysRS gene (Lanker et al., 1992). Presumably, this will maintain the optimum LysRS/tRNA^{Lys} ratio to keep all tRNA^{Lys} in a charged state. One could therefore predict that the cell might have a converse mechanism in which excess LysRS stimulates the synthesis of more tRNA^{Lys} to maintain the LysRS/tRNA^{Lys} ratio. However, because the increases in cytoplasmic tRNA^{Lys} concentration is induced by expression of mutant LysRS.T, which cannot enter the nucleus and does not bind well to tRNA^{Lys}, LysRS probably does not act directly on tRNA^{Lys} or its gene, but may instead bind another cellular factor which can alter either tRNA^{Lys} expression or stability.

Nevertheless, nuclear localization of LysRS must be necessary to fulfill some function other than directly modulating tRNA^{Lys} gene expression. Aminoacyl tRNA synthetases (aaRSs) have been found to be present in the nucleus (Hopper et al., 1998; Lund et al., 1998; Sarkar et al., 1999), and have been found there as high molecular weight aaRS complexes (Nathanson et al., 2000). Various functions for nuclear aaRSs have been proposed, including producing a more efficient export of aminoacylated tRNA from the nucleus (Lund et al., 1998; Sarkar et al., 1999) which may be part of a tRNA proof-reading mechanism, and the regulation of rRNA biogenesis in nucleoli (Ko et al., 2001). Nuclear localization signals (NLS) in aaRSs have been predicted (Schimmel et al., 1999), and our data suggests that LysRS may have an NLS within the first N-terminal 65 amino acids, since removal of this segment results in the loss of ability to migrate to the nucleus (FIG. 11).

The inability of LysRS.T to package tRNA^{Lys} is not to be confused with the normal presence of truncated LysRS in HIV-1. The presence of the intermediate sized LysRS fragment in virions has been correlated with tRNA^{Lys} incorporation into the viruses (Example 2). The modifications which produce the intermediate LysRS species have not yet been fully characterized, and might occur after viral packaging, i.e., not be related to packaging the tRNA^{Lys} per se, but rather be required to facilitate the annealing of primer tRNA^{Lys3} to the viral genome (e.g. releasing tRNA^{Lys3} so that it can interact with the retroviral genome). Furthermore, preliminary evidence using N- and C-terminal epitope tagging indicates that both C and N termini sequences are missing from the intermediate LysRS species (data not shown), i.e., the naturally-occurring viral intermediate fragment is not LysRS.T.

All detectable tRNA^{Lys} in the cell is aminoacylated (Huang et al., 1996), and it is assumed that almost all tRNA^{Lys} is associated with LysRS. Although our data indicate that LysRS is the limiting factor for tRNA^{Lys} viral incorporation, additional factors other than the total amount of LysRS in the cell may be involved. For example, a particular state of LysRS may be required for facilitating its interaction with Gag. In the mammalian cell, LysRS is part of a high molecular weight aminoacyl tRNA synthetase complex (HMW aaRS complex), which in addition to containing at least 8 other aaRSs, contains 3 non-synthetase proteins (Mirande et al., 1991). One of these, p38, appears to act as a scaffold for assembling the aaRSs, and LysRS is believed to bind first, and most tightly, to p38, and facilitate interaction with other components of the complex (Robinson et al., 2000). Since some of the components of this complex have already been found to be absent from HIV-1 (IleRS and ProRS (Example 2), the question remains whether LysRS in the HMW aaRS complex interacts with viral protein before or after release from the complex, or if instead, some LysRS which was not part of this HMW aaRS complex is the source used for viral packaging. The incorporation of LysRS.T in the virion does not contradict that HMW aaRS is the source of the enzyme since LysRS does not require the N terminus to interact with the HMW aaRS (Robinson et al., 2000). The cellular site of aminoacylation of the tRNA by aaRSs has also not been determined, and might occur away from the complex, with the complex acting primarily as an aaRS storage device. Overexpressed LysRS in the cell might result in the formation of a low molecular weight LysRS/tRNA^{Lys} complex which can interact with Gag. However, since LysRS.T is also packaged into the virions, interaction with Gag probably does not require the presence of tRNA^{Lys}.

### EXAMPLE 4

### Correlation Between tRNA^{Lys3} Aminoacylation and its Incorporation into HIV-1

The recognition and binding of aminoacyl tRNA synthetases (aaRSs) with their cognate tRNAs involves binding to the acceptor and/or anticodon arms of the tRNAs (Frugier et al., 2000; Schimmel et al., 1987). For human LysRS, sequences within the anticodon arm of tRNA^{Lys3} appear to play a more important role in binding LysRS than elements in the acceptor arm (Stello et al., 1999). Previous work has indicated that the anticodon sequence was not important for tRNA^{Lys} packaging into virions, i.e., not only do tRNA^{Lys3} (anticodon SUU, where S=^{mcm5s2}U) and tRNA^{Lys1,2} (anticodon CUU) appear to be packaged with equal efficiency, but we have reported that a mutant tRNA^{Lys3} with the anticodon CUA is also packaged efficiently (Huang et al., 1996). However, reports have indicated a relative insensitivity of in vitro tRNA^{Lys} aminoacylation to mutagenesis of anticodon nucleotides U34 and U36, compared to mutagenesis at U35, in both an in vitro E. coli system (Tamura et al., 1992) and an in vitro system using human LysRS and modified or unmodified human tRNA^{Lys3} (Stello et al., 1999). This agrees with previous findings that the tRNA^{Lys3} with the mutant anticodon CUA is still aminoacylated in vitro to 40% wild type levels (Huang et al., 1996).

Herein, we have constructed different tRNA^{Lys3} genes mutated in the anticodon region, and expressed these genes in COS7 cells also transfected with HIV-1 proviral DNA in order to assess their incorporation into HIV and hence their modulations of tRNA^{Lys3} priming processes. All mutant tRNA^{Lys3} molecules contain the mutation U35G, either alone or in combination with either the U34C or U36A mutations. We show that mutations in the tRNA^{Lys3} anticodon can strongly inhibit the interaction of LysRS with tRNA^{Lys3}, as manifest by the inhibition of aminoacylation in vivo. The order of decreasing aminoacylation for tRNA^{Lys3} anticodon mutants is: wild type UUU(100%)>UGU(49%)>CGU(40%)>UGA(0%)=CGA(0%). The ability of tRNA^{Lys3} to be aminoacylated in vivo is directly correlated with its ability to be incorporated into HIV-1.

Materials and Methods

Plasmid Construction

SVC21.BH10 is a simian virus 40-based vector which contain wild-type HIV-1 proviral DNA and obtained from E. Cohen, University of Montreal. SVC12.BH10^{Lys3} UUU contains the HIV-1 proviral DNA plus a wild type tRNA^{Lys3} gene. SVC12.BH10^{Lys3} CGA, SVC12.BH10^{Lys3} CGU, SVC12.BH10^{Lys3} UGU, and SVC12.BH10^{Lys3} UGA contain the HIV-1 proviral DNA plus a mutant tRNA^{Lys3} gene where the anticodon has been changed from TTT to CGA, CGT, TGT and TGA respectively. Mutant tRNA^{Lys3} genes were created by PCR mutagenisis (Huang et al., 1994). The amplified products were cloned into the Hpa-I site of SVC21.BH10, which is upstream of the HIV-1 proviral DNA sequence. Mutations were confirmed by DNA sequencing.

Production of Wild-Type and Mutant HIV-1 Virus

Transfection of COS7 cells with the above plasmids by the calcium phosphate method was as previously described (Mak et al., 1997). Viruses were isolated from COS7 cell culture medium 63 h posttransfection, or from the cell culture medium of infected cell lines. The virus-containing medium was first centrifuged in a Beckman GS-6R rotor at 3,000 rpm for 30 minutes and the supernatant was then filtered through a 0.2 μm filter. The viruses in the filtrate were then pelleted by centrifugation in a Beckman Ti45 rotor at 35,000 rpm for 1 h. The viral pellet was then purified by centrifugation with a Beckman SW41 rotor at 26,500 rpm for 1 h through 15% sucrose onto a 65% sucrose cushion.

RNA Isolation and Analysis

Total cellular or viral RNA was extracted from cell or viral pellets by the guanidinium isothiocyanate procedure (Dufour et al., 1999), and dissolved in 5 mM Tris buffer, pH 7.5. Hybridization to dot blots of cellular or viral RNA were hybridized with DNA probes complementary to tRNA^{Lys3} and tRNA^{Lys1,2} (Khorchid et al., 2000), genomic RNA (Example 2), and ∃ actin mRNA (DNA probe from Ambion). 2D PAGE of 32pCp-3′ end labeled viral RNA was carried out as previously described (Khorchid et al., 2000).

Measurement of Wild Type and Mutant tRNA^{Lys3} Using RNA-DNA Hybridization

To measure the amount of tRNA^{Lys3} (wild type and mutant) present in cellular or viral RNA, we have synthesized an 18-mer DNA oligonucleotide complimentary to the 3′ 18 nucleotides of tRNA^{Lys3}-(5′ TGGCGCCCGMCAGGGAC 3′). This probe has previously been shown to hybridize specifically with tRNA^{Lys3} (Khorchid et al., 2000), and was hybridized to dot blots on Hybond N (Amersham) containing known amounts of purified in vitro transcript of tRNA^{Lys3} and either cellular tRNA or viral RNA produced in cells transfected with either SVC21.BH10 alone, or SVC21.BH10 containing a wild type or mutant tRNA^{Lys3} gene. The DNA oligomer was first 5′-end labeled using T4 polynucleotide kinase and gamma-³²P-ATP (3000 Ci/mMol, Dupont Canada), and specific activities 10⁸ to 10⁹ cpm/ug were generally reached. Approximately 10⁷ cpm of oligomer was generally used per blot in hybridization reactions.

For detection of specific wild type or mutant tRNA^{Lys3}, DNA probes complementary to the anticodon arm were used (see FIG. 7): wild type tRNA^{Lys3}UUU, (5′CCCTCAGATTAAAAGTCTGATGC3′); tRNA^{Lys3}CGA, (5′CCCTCAGATTTCGAGTCTGATGC-3′); tRNA^{Lys3} CGU, (5′CCCTCAGATTACGAGTCTGATGC-3′); tRNA^{Lys3}UCU, (5′CCCTCAGATTACMGTCTGATGC-3′); and tRNA^{Lys3}UCA, (5′CCCTCAGATTTCAAGTCTGATGC-3′). In order to specifically detect the presence of tRNA^{Lys3} mutants in RNA samples, blots were hybridized with ³²P labelled anticodon probes to the tRNA^{Lys3} mutants in the presence of 8-25 fold excess of non-radioactive oligonucleotide complementary to the wild type tRNA^{Lys3} anticodon arm.

Measurement of In Vivo Aminoacylation

In vivo aminoacylation of tRNA^{Lys} was measured using techniques previously described (Ho et al., 1986; Huang et al., 1994; and Varshney et al., 1991). To measure the extent of in vivo aminoacylation of tRNA^{Lys3}, the isolation of cellular or viral RNA was performed at low pH conditions required for stabilizing the aminoacyl-tRNA bond. The guanadinium thiocyanate procedure for isolating RNA[5] was modified by including 0.2M sodium acetate, pH 4.0 in solution D, and the phenol used was equilibrated in 0.1M sodium acetate, pH 5.0. The final isopropanol-precipitated RNA pellet was dissolved in 10 mM sodium acetate, pH 5.0, and stored at −70EC until electrophoretic analysis. RNA was mixed with one volume loading buffer (0.1 M sodium acetate, pH 5.0, 8 M urea, 0.05% bromphenol blue, and 0.05% xylene cyanol), and electrophoresed in a 0.5 mm thick polyacrylamide gel containing 8 M urea in 0.1 M sodium acetate, pH 5.0. The running buffer was 0.1 M sodium acetate, pH 5.0, and electrophoresis was carried out at 300 V, at 4° C., for 15-18 hours in a Hoefer SE620 electrophoretic apparatus. RNA was electroblotted onto a Hybond N filterpaper (Amersham) using an electrophoretic transfer cell (Bio-Rad) at 750 mA for 15 min, using 1×TBE. Hybridization of the blots with probes for wild type and mutant tRNA^{Lys3} were performed as described above. Deacylated tRNA was produced by treating the RNA sample with 0.1 M Tris-HCl, pH 9.0 at 37° C. for 3 hours to hydrolyze the aminoacyl linkage and provide an uncharged electrophoretic marker.

Western Blotting

Western blot analysis was performed using 300 μg of cytoplasmic or nuclear proteins, as determined by the Bradford assay (Barat et al., 1989). Cytoplasmic and nuclear extracts were resolved by SDS-PAGE followed by blotting onto nitrocellulose membranes (Gelman Sciences). Detection of protein on the Western blot utilized monoclonal antibodies (anti YY1). Western blots were analyzed by enhanced chemiluminescence (ECL kit, Amersham Life Sciences) anti-mouse (Amersham Life Sciences) as a secondary antibody. The sizes of the detected protein bands were estimated using pre-stained high molecular mass protein markers (GIBCO/BRL).

Cell Fractionation

The cytoplasmic supernatant and nuclear extract were prepared from the COS7 cells as described previously (Mak et al., 1997). Western blot analysis was performed as above using anti-YY1 (Santa Cruz).

Results

Expression of Wild Type and Mutant tRNA^{Lys3} and Their Incorporation into Virions

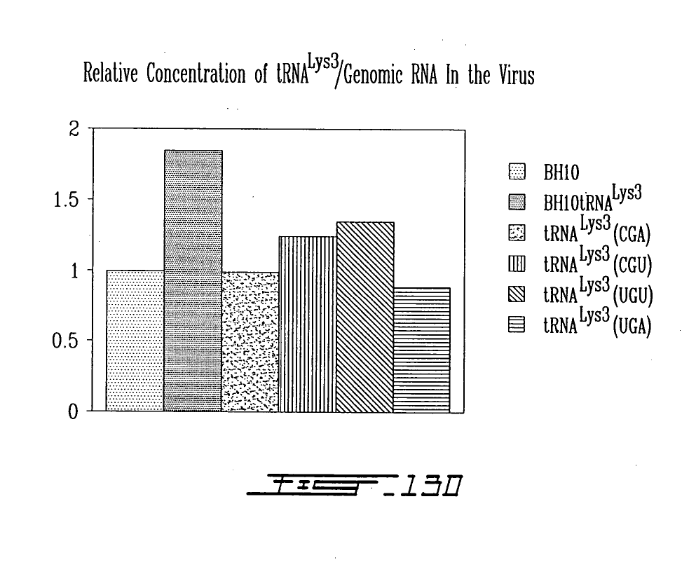

We have determined whether a correlation exists between the ability of a tRNA to be aminoacylated in vivo and to be incorporated into HIV-1. COS7 cells were transfected with a plasmid containing both HIV-1 proviral DNA and a wild type or mutant tRNA^{Lys3} gene. As shown previously, this results in more tRNA^{Lys3} being synthesized in the cytoplasm and being packaged into the viruses (Huang et al., 1994). The ability of tRNA^{Lys} to be aminoacylated in vitro was shown to be most sensitive to sequences in the anticodon, and in particular, to U35 (Stello et al., 1999). Therefore, the different mutant tRNA^{Lys3} being expressed all contained a U35G transition, in addition to other possible anticodon mutations (U34C or U36A—see FIG. 12).

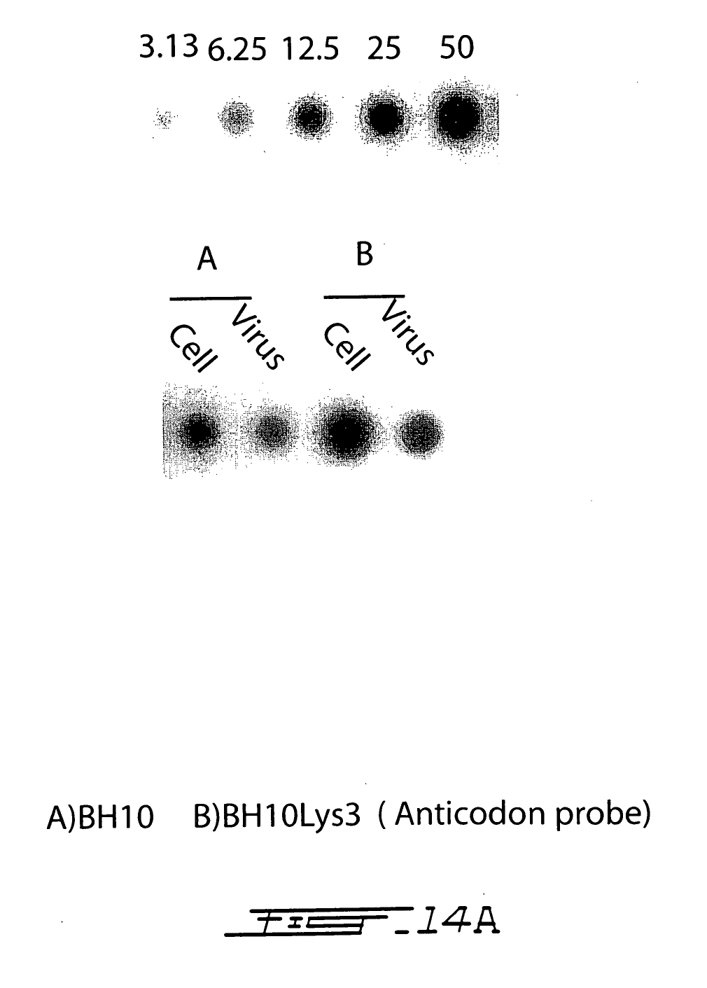

FIG. 13A shows dot blots of cellular or viral RNA hybridized with a radioactive 18 nucleotide DNA oligomer complementary to the 3 terminal 18 nucleotides of tRNA^{Lys3}. The top panel represents increasing amounts of synthetic tRNA^{Lys3}, and the hybridization results are plotted as a standard curve in FIG. 13C. The bottom 2 panels in FIG. 13A show dot blots of RNA isolated from either cell lysate containing equal amount of b actin (cell) or viral lysates containing equal amounts of viral genomic RNA (viral). Western blots for determining 3 actin amounts, and dot blots for determining genomic RNA amounts, are not shown. The relative total tRNA^{Lys3}/∃ actin ratios are plotted in FIG. 13B, normalized to the value obtained in COS7 cells transfected with HIV-1 proviral DNA alone (BH 10). Transfection with the wild type tRNA^{Lys3} gene or the mutant tRNA^{Lys3} genes results in an approximate two fold increase in the cytoplasmic concentration of total tRNA^{Lys3}. However, as shown in FIG. 13D, these cytoplasmic increases in tRNA^{Lys3} did not all result in increases in tRNA^{Lys3} incorporation into virions. The maximum increase in tRNA^{Lys3} incorporation into virions occurred with excess wild type tRNA^{Lys3}_{UUU} (1.85). tRNA^{Lys3}_{UGU} and tRNA^{Lys3}_{CGU} increased 1.4 and 1.3, respectively. tRNA^{Lys3}_{CGA} showed no increase in tRNA^{Lys3} incorporation, and tRNA^{Lys3}_{UGA} actually showed a small decrease in packaging compared to wild type tRNA^{Lys3}_{UUU}.

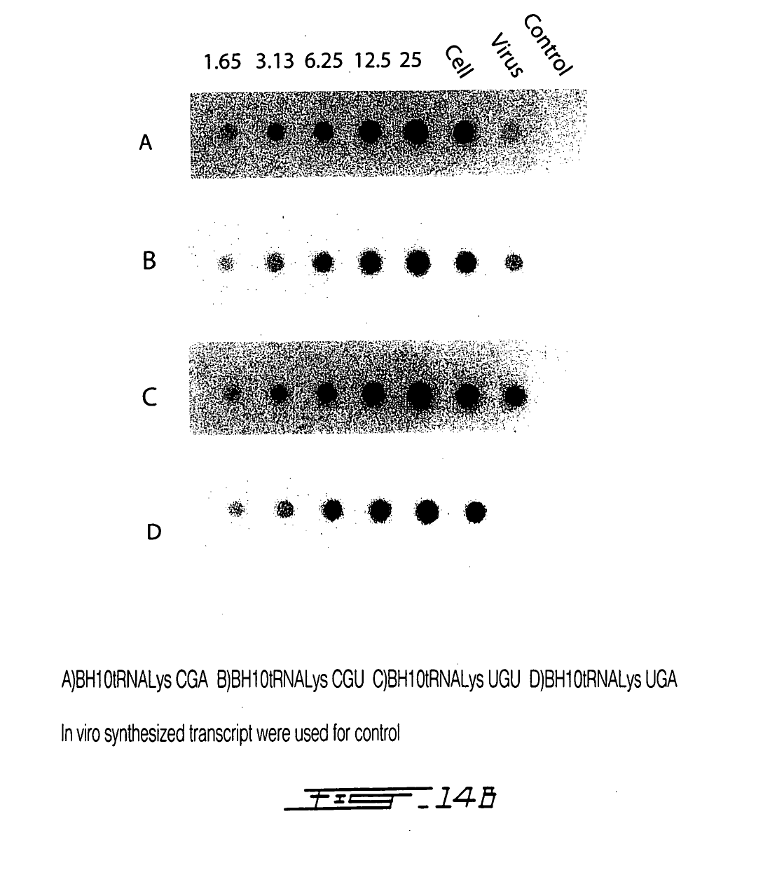

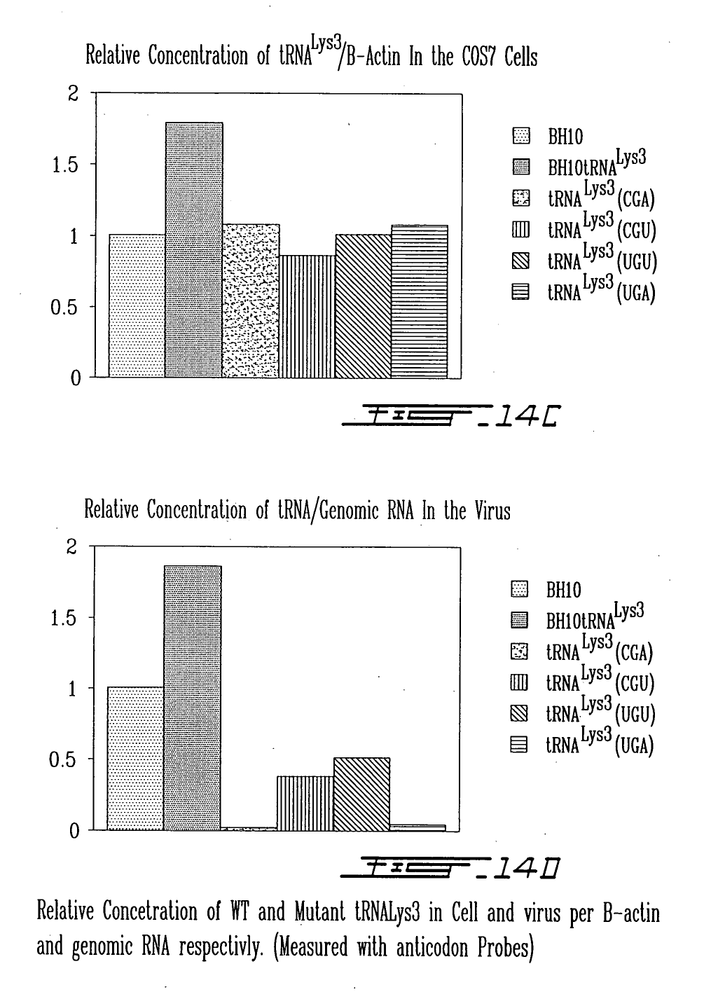

The experiments in FIG. 13 measure total tRNA^{Lys3} in the cytoplasm and in the virion. We have also used anticodon hybridization probes specific for each type of tRNA^{Lys3} to examine their expression in the cytoplasm and incorporation into virions. This is shown in FIG. 14. The dot blots in panel A, which measure the amount of a specific tRNA^{Lys3} present in cell or viral lysate, use RNA from cell or viral lysates containing equal amounts of 3 actin or genomic RNA, respectively. For each type of RNA, a standard hybridization curve is generated using synthetic mutant tRNA^{Lys3} transcripts. FIG. 14A shows the amount of tRNA^{Lys3} in cytoplasm and in viruses in cells transfected with HIV-1 alone (BH10) or transfected with HIV-1 and a tRNA^{Lys3} gene (BH10^{Lys3}). FIG. 14B shows the amount of tRNA^{Lys3} in cytoplasm and viruses for cells transfected with HIV-1 and a mutant tRNA^{Lys3} gene. In FIG. 14B, the wild type tRNA^{Lys3} transcript was used as a control for specific hybridization of the anticodon probes. The standard curves for each type of tRNA^{Lys3} are used to calculate ngms present in cell lysate or virus, and thereby taking into account any differences in efficiencies of hybridization which the different anticodon probes might have.

The relative total tRNA^{Lys3}/∃ actin ratios are plotted in FIG. 14C, normalized to the value found for cells transfected with HIV-1 alone (BH10). The results are very similar to that shown in FIG. 13 using a DNA hybridization probe which measures total tRNA^{Lys3} (wild type and mutant). In this preparation, wild type tRNA^{Lys3} is increased significantly when cells are transfected with a wild type tRNA^{Lys3} gene, although not quit as much as shown in the preparation in FIG. 13. Expression of each mutant tRNA^{Lys3} in the cytoplasm are similar, and would result in an approximate 2 fold increase in total tRNA^{Lys3} (endogenous wild type and mutant), which was shown in FIG. 13. The tRNA^{Lys3}/genomic RNA ratios in virions are shown in FIG. 14D, normalized to the value found for cells transfected with HIV-1 alone (BH10), and also match with similar results for total viral tRNA^{Lys 3} shown in FIG. 13. Wild type tRNA^{Lys3}_{UUU} incorporation into virions increased the ratio to 1.87, indicating a relative incorporation of exogenous tRNA^{Lys3} compared to endogenous tRNA^{Lys3} of 0.87. The relative incorporation of tRNA^{Lys3}_{UGU} and tRNA^{Lys3}_{CGU} was 0.50 and 0.37, respectively, while tRNA^{Lys3}_{CGA} and tRNA^{Lys3}_{UGA} showed relative incorporations of 0.013 and 0.29.

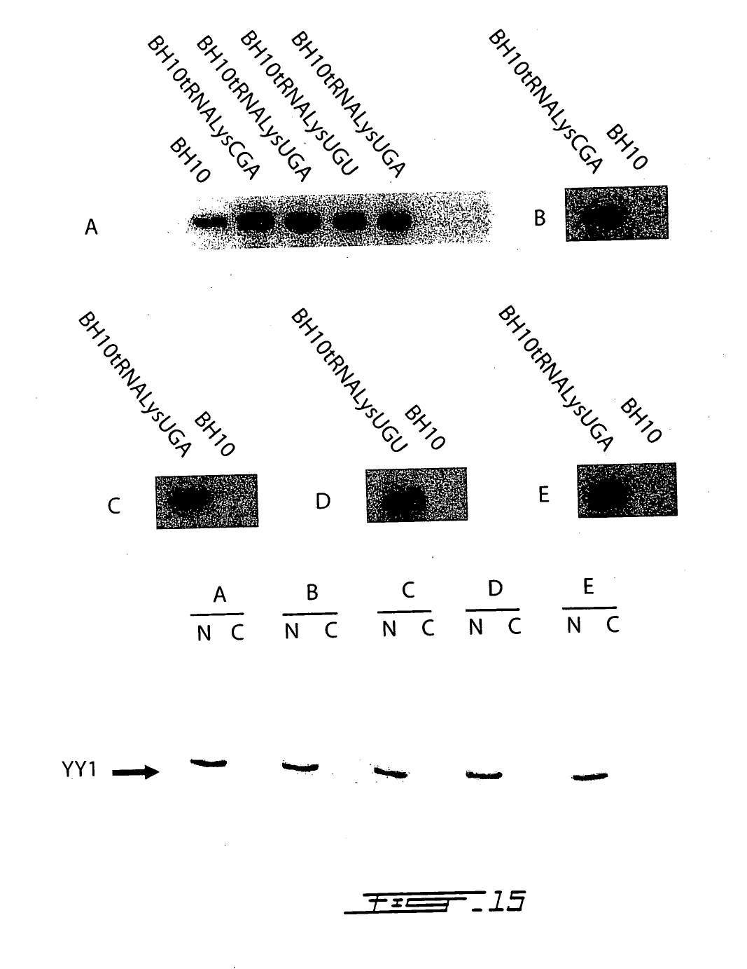

These data indicate that wild type or mutant tRNA^{Lys3} are expressed at approximately equal levels in the total cell lysate, but some mutant tRNAs are not incorporated into virions as well as others. One possible explanation could be that some mutant tRNAs are not exported with equal efficiency out of the nucleus. To test this we lysed cells, and separated nuclei from cytoplasm by low speed centrifugation. Dot blots of the RNA in cytoplasmic fraction, representing equal amouts of b actin, were hybridized with either the 3′ terminal DNA probe, which hybridizes to all tRNA^{Lys3}s (FIG. 15A) or with anticodon probes specific for each tRNA^{Lys3} (FIGS. 15B-E). In panels B-E, RNA from the BH10 cytoplasmic fraction was used as the control to show hybridization specificity for each anticodon probe. It can be seen that, as concluded in FIG. 15 which used total cell lysates, that all tRNA^{Lys3}s are expressed approximately equally. Panel F at the bottom of the figure demonstrates the efficiency of the separation of nuclear and cytoplasmic fractions, ie, the nuclear transcription factor YYI, which concentrates in the nucleus, is only detected in that fraction.

Aminoacylation of Wild Type and Mutant tRNA^{Lys3} In Vivo

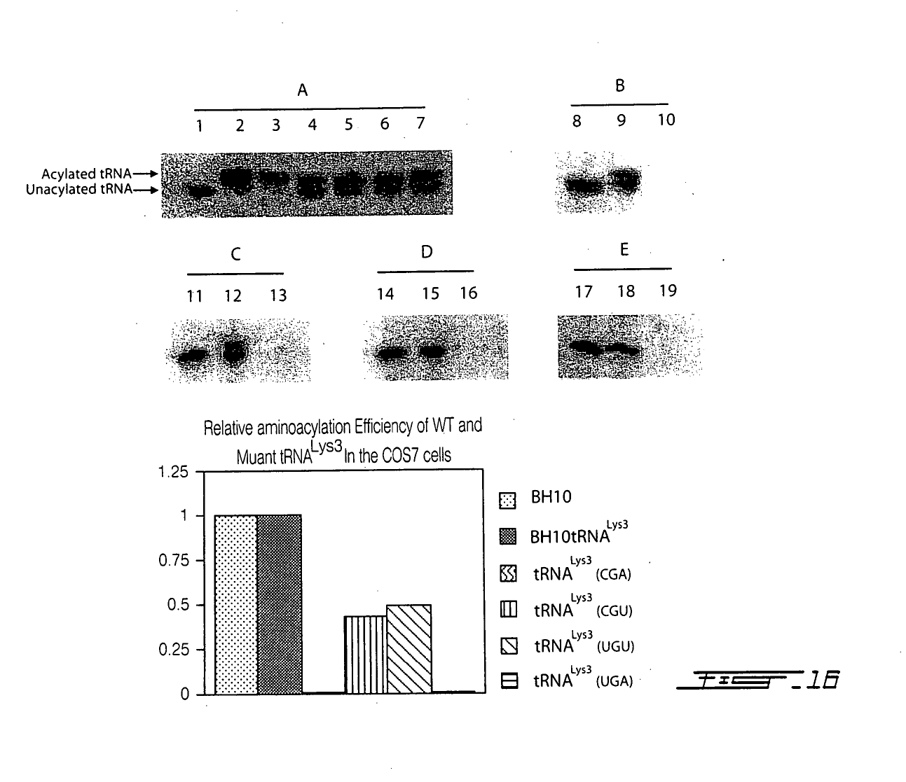

The aminoacylation state of the wild type and mutant cellular tRNA^{Lys3} were examined. The electrophoretic mobility of acylated tRNA in acid-urea PAGE has been reported to be slower than the deacylated form, and this property can be used to determine the degree of tRNA aminoacylation (Huang et al., 1996). FIG. 16 shows northern blots of cellular and viral RNA samples electrophoresed in acid-urea gels, blotted onto Hybond Nl filterpaper, and hybridized with radioactive tRNA^{Lys3} DNA probes. In panel A, cellular tRNA was hybridized with the 18 nucleotide DNA oligomer complementary to the 3′ 18 nucleotide terminus of tRNA^{Lys3}, while in panels B-E, the cellular tRNA was hybridized with the anticodon probes specific for different mutant tRNAs. Lane 1 in panel A represents wild type tRNA^{Lys3} deacylated in vitro to mark where deacylated tRNA^{Lys3} migrates in the gel. As has previously been reported (Huang et al., 1996), in cells transfected with either the wild type tRNA^{Lys3} gene (lane 2), or not transfected with any tRNA^{Lys3} gene (lane 3), the tRNA detected is entirely in the aminoacylated form. This is shown graphically in panel F, where cytoplasmic aminoacylation is given as 100%. It can also be seen in panel A that a majority of the total tRNA^{Lys3} is aminoacylated in cells transfected with genes coding for tRNA^{Lys3}_{CGU} (lane 5) and tRNA^{Lys3}_{UGU} (lane 6), with a larger proportion of total tRNA^{Lys3} being in the deacylated form in cells transfected with genes coding for tRNA^{Lys3}_{CGA} (lane 4) and tRNA^{Lys3}_{UGA} (lane 7).

Since total tRNA^{Lys3} consists of both endogenous tRNA^{Lys3}_{UUU} and exogenous mutant tRNA^{Lys3}, the data in panel A gives us an indirect view of the ability of the mutant tRNA^{Lys3}s to be aminoacylated. We therefore used anticodon DNA probes specific for the different mutant tRNA^{Lys3}s (panels B-E). Lanes 8,11,14, and 17 represent the corresponding mutant tRNA^{Lys3} samples which have been deacylated in vitro, while lanes 10, 13, 16, and 19 contain cellular RNA from cells transfected only with HIV-1 proviral DNA, and show the hybridization specificity of the anticodon probes. It is clear from the data in these panels that tRNA^{Lys3}_{UGU} (lane 9) and tRNA^{Lys3}_{CGU} (lane 12) are aminoacylated better than tRNA^{Lys3}_{CGA} (lane 15) and tRNA^{Lys3}_{UGA} (lane 18). The percentage of each mutant tRNA^{Lys3} which is aminoacylated is also shown graphically in panel F.

Discussion

Herein, we have shown that the ability of tRNA^{Lys3} to be incorporated into HIV-1 is closely correlated with its ability to be aminoacylated. Aminoacylation is dependent upon the binding of LysRS to tRNA^{Lys3}, demonstrating that this interaction is required for tRNA^{Lys} incorporation into virions. Whether aminoacylation itself is required for viral tRNA^{Lys} packaging cannot be inferred from this data. Other data presented herein is consistent with LysRS binding to tRN^{Lys} playing an important role in tRNA^{Lys} packaging, however. For example, when COS7 cells are cotransfected with plasmids containing both HIV-1 proviral DNA and a LysRS gene, the viral tRNA^{Lys} concentration goes up 2 fold. On the other hand, transfection with a mutant, N-terminally truncated LysRS gene, which produces LysRS unable to bind to tRNA^{Lys}, does not result in any increase in tRNA^{Lys} packaging, although the mutant LysRS is still packaged into the virion (Example 2).

The data presented in this work supports a model in which the tRNA^{Lys3}/LysRS interaction is important for tRNA^{Lys3} incorporation into viruses. However, the anticodon sequence has also been implicated in the in vitro binding of mature reverse transcriptase to either purified tRNA^{Lys3} (Sarih-Cottin et al., 1992) or tRNA^{Lys3} transcripts (Barat et al., 1989; Wohrl et al., 1993). Since RT sequences in GagPol have been implicated in an interaction with tRNA^{Lys3} during it incorporation into virions (Khorchid et al., 2000; Mak et al., 1994), mutant anticodon might also weaken this tRNA^{Lys3}/Gag^{Pol} interaction. Both in vitro studies (Dufour et al., 1999; Mishima et al., 1995) and in vivo studies (Khorchid et al., 2000) indicate that the thumb domain sequences within RT probably interact with tRNA^{Lys3}. In vitro cross linking studies indicate an interaction between RT peptides containing the thumb domain and either synthetic (Mishima et al., 1995) or purified (Dufour et al., 1999) tRNA^{Lys3}. In vivo, it has been shown that tRNA^{Lys3} incorporation into HIV-1 is not affected by deletion of the IN domain in Pr160^{gag-pol}, nor by further deletion of the RNaseH and connection subdomains within the RT domain of Pr160^{gag-pol}. However, tRNA^{Lys3} packaging is severely inhibited by further deletions into the thumb subdomain (Khorchid et al., 2000).

However, the site of RT interaction on the tRNA^{Lys3} is in question. While one report indicates an interaction in vitro between the thumb domain and the tRNA^{Lys3} anticodon loop (Mishima et al., 1995), another report indicates an interaction in vitro between the thumb domain and the 3′ terminus of tRNA^{Lys3} (Dufour et al., 1999). These differences may be due to the use of synthetic tRNA^{Lys3} in the former case, and purified tRNA^{Lys3} in the latter case. Using mutational analysis, Arts et al., (Arts et al., 1998) also found evidence for an in vitro interaction between the anticodon loop of tRNA^{Lys3} and a small crevice in the p66 thumb domain of RT. Herein, mutations in certain amino acids in the thumb subdomain (K249, R307) were found to inhibit the interaction of mature RT with the tRNA^{Lys3} anticodon domain in vitro. However, since these same RT amino acid mutations had no effect upon tRNA^{Lys3} packaging in vivo (Khorchid et al., 2000), there is no evidence for an in vivo interaction between RT sequences in GagPol and the tRNA^{Lys3} anticodon during tRNA^{Lys3} packaging.

Conclusion

In summary, the present invention shows a positive correlation between the amount of tRNA^{Lys3} incorporated into virions, the amount of tRNA^{Lys3} annealed to the viral genome, and the infectivity of HIV virions. Furthermore, the tRNA^{Lys}-binding protein, lysyl-tRNA synthetase (LysRS), is selectively packaged along with tRNA^{Lys} into HIV-1, and the amount of LysRS in the virus determines the amount of tRNA^{Lys} packaged into the virus. In addition, the ability of tRNA^{Lys3} to be incorporated into HIV-1 or to affect LysRS-facilitated processes associated with tRNA^{Lys3} priming of RT, is shown to be closely correlated with its ability to be aminoacylated, and hence of its binding to LysRS. The viral precursor protein Pr55^{gag} alone will package LysRS into Pr55^{gag} particles, independently of tRNA^{Lys}. With the additional presence of the viral precursor protein Pr160^{gag-pol}, tRNA^{Lys} and LysRS are both packaged into the particle. While the predominant cytoplasmic LysRS has an apparent Mr=70,000, viral LysRS associated with tRNA^{Lys} packaging is truncated and has an apparent Mr=63,000.

Although the present invention has been described hereinabove by way of preferred embodiments thereof, it can be modified without departing from the spirit and nature of the subject invention as defined in the appended claims.

### REFERENCES

Arts et al., 1998, Journal of Biological Chemistry 273:14523-32.

Barat et al., 1989, EMBO J. 8:3279-3285.

Berkowitz et al., 1996, RNA packaging, p. 177-218. In H. G. Krausslich (ed.), Morphogenesis and maturation of retroviruses., vol. 214. Springer-Verlag, Berlin Heidelberg N.Y.

Bradford M. M., 1976, Anal. Biochemistry 72:248-254.

Bruce et al., 1978, Nucleic Acids Research 5:3665-3677.

Cen et al., 1999, J. Virol. 73:4485-8.

Cerini et al., 1991, EMBO J. 10:4267-4277.

Chomczynski et al., 1987, Analytical Biochemistry 162:156-159.

Cimarelli et al., 1999, J. Virol. 73:5388-5401.

Ciracoglu et al., 1985, Eur. J. Biochem. 149:353-361.

Cirakoglu et al., 1985, Eur. J. Biochem. 151:101-110.

Dettenhofer et al., 1999, J. Virol. 73:1460-7.

Dufour et al., 1999, Journal of Molecular Biology. 285:1339-46.

Entelis et al., 1998, Proc. Natl. Acad. Sci. USA. 95:2838-2843.

Faras et al., 1975, Proc. Natl. Acad. Sci. USA. 72:859-863.

Feng et al., 1999, J. Virol. 73:4251-6.

Frugier et al., 2000, The EMBO J. 19:2371-2380.

Fu et al., 1997, J. Virol. 71:6940-6946.

Harada et al., 1975, J. Biol. Chem. 250:3487-3497.

Harada et al., 1979, J. Biol. Chem. 254:10979-10985.

Heacock et al., 1996, Bioorganic Chem. 24:273-289.

Ho et al., 1987, Proc. Natl. Acad. Sci. USA 84:2185-2188.

Hopper A. K., 1998, Science 282:2003-2004.

Huang et al., 1994, J. Virol. 68:7676-7683.

Huang et al., 1996, J. Virol. 70:4700-4706.

Huang et al., 1997, J. Virol. 71:4378-4384.

Jiang et al., 1993, J. Virol. 67:3246-3253.

Khorchid et al., 2000, J. Mol. Biol. 299:17-26.

Kim et al., 2000, Nucl. Acid. Res. 28:2866-2872.

Kimpton et al., 1992, J. Virol. 66:2232-2239.

Ko et al., 2001, J. Cell Biol. 149:567-574.

Lanker et al., 1992, Cell 70:647-657.

Leis et al., 1993. Regulation of Initation of Reverse Transcription of Retroviruses, p. 33-47. In A. M. Skalka, and S. P. Goff (eds), Reverse Transcriptase, vol. 1. Cold Spring Harbor Laboratory Press, New York, N.Y.

Levin et al., 1979, J. Virol. 29:328-335.

Levin et al., 1981, J. Virol. 38:403-408.

Levin et al., 1984, J. Virol. 51:470-478.

Lund et al., 1998, Science 282:2082-2085.

Mak et al., 1994, J. Virol. 68:2065-2072.

Mak et al., 1997, J. Mol. Biol. 265:419-431.

Mak et al., 1997, J. Virol. 71:8087-8095.

Meinnel et al., 1995, Aminoacyl-tRNA synthetases: Occurrence, structure, and function., p. 251-292. In D. Soll, and U. L. RajBhandary (eds), tRNA: Structure, Biosynthesis, and Function. ASM Press, Washington, D.C.

Mirande et al., 1988, J. Biol. Chem. 263:18443-18451.

Mirande M., 1991, Progress in Nuclei Acid Research and Molecular Biology 40: 95-142.

Mishima et al., 1995, EMBO J. 14:2679-2687.

Nathanson 2000, J. Biol. Chem. 275:31559-31562.

Ott et al., 1995, AIDS Research and Human Retroviruses. 11:1003-1006.

Park et al., 1992, J. Virol. 66:6304-6313.

Peters et al., 1977, J. Virol. 21:1031-1041.

Peters et al., 1980, J. Virol. 35:31-40.

Peters et al., 1980, J. Virol. 36:692-700.

Prats et al., 1988, EMBO J. 7:1777-1783.

Raba et al., 1979, Eur. J. Biochem. 97:305-318.

Robinson et al., 2000, J. Mol. Biol. 304:983-994.

Sarih-Cottin et al., 1992, J. Mol. Biol 226:1-6.

Sarkar et al., 1999, Proc. Natl. Acad. Sci. USA 96:14366-14371.

Sawyer et al., 1973, J. Virol. 12:1226-1237.

Schimmel et al., 1999, TIBS. 24:127-128.

Schimmel P., 1987, Annu. Rev. Biochem. 56:125-158.

Shiba et al., 1994, Proc. Natl. Acad. Sci. USA. 91:7435-7439.

Shiba et al., 1997, J. Biol. Chem. 272:22809-22816.

Smith et al., 1990, J. Virol. 64:2743-50.

Smith et al., 1993, J. Virol. 67:2266-2275.

Stapulionis et al., 1995, Proc. Natl. Acad. Sci. USA. 92:7158-7161.

Stark et al., 1998, J. Virol. 72:3037-3044.

Stello et al., 1999, Nucl. Acid. Res. 27:4823-4829.

Swanstrom et al., 1997, Synthesis, assembly, and processing of viral proteins., p. 263-334. In J. Coffin, S. Hughes, and H. Varmus (eds), Retroviruses. Cold Spring Harbor Laboratory Press, Plainview, N.Y.

Tamura et al., 1992, Nucleic Acids Res. 20:2335-2339.

Tarassov et al., 1995, The EMBO J. 14:3461-3471.

Taylor, J. M., 1977, Biochim. Biophys. Acta. 47:57-71.

Ting et al., 1992, J. Biol. Chem. 267:17701-17709.

Tolkunova et al., 2000, J. Biol. Chem. 275:35063-35069.

Varshney et al., 1991, J. Biol. Chem. 266:24712-24718.

Wakasugi et al., 1999, Science. 284:147-150.

Waters et al., 1975, Proc. Natl. Acad. Sci. USA. 72:2155-2159.

Waters et al., 1977, Prog. Nucleic Acid Res. Mol. Biol. 20:131-160.

Waters, L. C., 1978, Biochem. Biophys. Res. Commun. 81:822-827.

Wöhrl et al., 1993, J. Biol. Chem. 268:13617-13624.

Zhang et al., 1996, Virology 226:306-317.

What is claimed is:

1. A method of modulating the incorporation of a tRNA involved in reverse transcriptase (RT) priming into a retroviral virion, comprising a modulation of the activity and/or of the level of a cognate aminoacyl tRNA synthetase, wherein the level and/or activity of said cognate aminoacyl tRNA synthetase in a cell infected by said retrovirus positively correlates with an incorporation of said tRNA into said virion.

2. The method of claim 1, wherein said tRNA is tRNA^{Lys3}, said aminoacyl tRNA synthetase is LysRS and said retroviral virion is HIV or SIV.

3. A method of targeting a molecule into a retrovirus virion comprising providing said molecule linked to a sufficient number of aminoacyl tRNA synthetase involved in transporting its cognate tRNA into a retroviral vi don of said retrovirus in a cell infected with said retrovirus, whereby incorporation of said aminoacyl tRNA synthetase into said virion enables incorporation of said molecule thereinto.

4. The method of claim 3, wherein said retrovirus is HIV and said aminoacyl tRNA synthetase is LysRS.

5. A chimeric protein capable of being incorporated into HIV or SIV virions, comprising a first and second portion, wherein said first portion comprises a sufficient number of amino acids of an intermediate form of LysRS to enable incorporation of said chimeric protein into said virions.

6. The chimeric protein of claim 5, wherein said retrovirus is HIV and said aminoacyl tRNA transferase is LysRS.

7. The chimeric protein of claim 5, wherein said second portion is a polypeptide covalently attached to said first portion.

8. The chimeric protein of claim 6, wherein said polypeptide fragment comprises an amino acid sequence having an antiviral activity.

9. The chimeric protein of claim 8, wherein said polypeptide fragment comprises an amino acid sequence which prevents proper virion morphogenesis of said HIV or SIV virions.

10. A molecule for interfering with incorporation of a native tRNA involved in reverse transcriptase (RT) priming and/or of its native cognate aminoacyl tRNA synthetase into a retroviral virion, wherein said molecule is expressed in trans with respect to the retroviral genome and comprises one of:
a) an aminoacyl tRNA synthetase incorporation domain;
b) said tRNA molecule involved in RT priming or a variant thereof, and
c) a precursor protein of said retroviral virion;
and wherein said molecule interferes with said incorporation of said tRNA and/or said aminoacyl/tRNA synthetase, into said virion, thereby reducing the infectivity of said retroviral virion.

11. The molecule of claim 10, wherein said tRNA is tRNA^{Lys}, said aminoacyl tRNA synthetase is LysRS, said precursor protein selected from Pr55^{gag} and Pr160^{gag} and said retroviral virion is HIV or related viruses.

12. The molecule of claim 11, wherein said native tRNA involved in RT is tRNA^{Lys3} and said HIV is HIV-1.

13. A method of screening and selecting an agent that modulates the incorporation of a tRNA and/or a cognate aminoacyl tRNA synthetase thereof into a retroviral virion comprising:
a) incubating a candidate agent with a cell expressing at least a portion of said aminoacyl tRNA synthetase, said portion being sufficient for enabling incorporation into said virion;
wherein said cell also contains said retroviral virion, such that said aminoacyl tRNA synthetase is capable of being incorporated into said virion; and
b) determining the amount of said aminoacyl tRNA synthetase and/or said tRNA incorporated into said virions;
wherein an agent that modulates the incorporation of said aminoacyl tRNA synthetase and/or tRNA into said virion is selected when the amount of incorporated aminoacyl tRNA synthetase and/or said tRNA in the presence of said candidate agent is measurably different than in the absence thereof.

14. The method of claim 13, wherein said tRNA is tRNA^{Lys}, said aminoacyl tRNA synthetase is LysRS and said retroviral virion is HIV or related viruses.

15. The method of claim 14, wherein said tRNA is tRNA^{Lys3} and said incorporation of said tRNA^{Lys3} into said virion is assessed by measuring RT priming function.

16. A method for reducing the infectivity of a retrovirus, comprising a reduction in the incorporation of a tRNA involved in RT priming and/or of the cognate aminoacyl tRNA synthetase thereof.

17. The method of claim 16, wherein said tRNA is tRNA^{Lys3}, said aminoacyl tRNA synthetase is LysRS and said retroviral virion is HIV or related viruses.

18. A method of modulating an aminoacyl tRNA synthetase-facilitated process associated with its cognate tRNA priming function of reverse transcriptase (RT) wherein this process is selected from the group consisting of a) cognate tRNA incorporation into the retrovirus virion; b) annealing thereof to the primer binding site (PBS) or other retroviral RNA regions; and c) initiation of RT, comprising a modulation of the activity and/or of the level of said cognate aminoacyl tRNA synthetase, a modulation of said cognate tRNA-aminoacyl tRNA synthetase interaction, a modulation of aminoacyl tRNA-Gag interaction, or a modulation of aminoacylation of cognate tRNA, wherein the level and/or activity of said cognate aminoacyl tRNA synthetase, or aminoacylation level of said cognate tRNA in a cell infected by said retrovirus positively correlates with at least one of a) an incorporation of said tRNA into the virion; b) the placement of said tRNA onto the retroviral genome; and c) infectivity of the retrovirus.

19. A method of screening and selecting an agent that modulates the incorporation of a tRNA and/or a cognate aminoacyl tRNA synthetase thereof into a retroviral virion comprising: a) incubating a candidate agent with a cell expressing at least a portion of the aminoacyl tRNA synthetase, said portion being sufficient for enabling incorporation into the virion; wherein the cell also contains the retroviral virion, such that the aminoacyl tRNA synthetase is capable of being incorporated into the virions; and b) determining one of the amount of the aminoacyl tRNA synthetase incorporated into the virion; the amount of said cognate tRNA incorporated into the virion; and the amount of reverse transcriptase (RT) priming in said virion; wherein an agent that modulates the incorporation of the aminoacyl tRNA synthetase and/or tRNA into the virion is selected when the amount of incorporated aminoacyl tRNA synthetase, cognate tRNA, or the level of RT priming in the presence of the candidate agent is measurably different than in the absence thereof.


SEQUENCE LISTING (4 sequences)

SEQ ID NO: 1 - RNA, 76 nt, Homo sapiens
  misc_feature (10)..(10): N= m2G
gcccggcuancucagncggnagagcangngacucuunancncaggnnngugggnncgngccccacguugggcgcca

SEQ ID NO: 2 - RNA, 76 nt, Homo sapiens
  misc_feature (10)..(10): N = m2G
gcccggauancucagncggnagagcancagacunuunancugaggnnnnagggnncangucccuguucgggcgcca

SEQ ID NO: 3 - RNA, 76 nt, Homo sapiens
  misc_feature (29)..(29)
  misc_feature (29)..(29): n is a, c, g, or u
gcccggcuagcucagucgguagagcaugngacucuuaaucncagggucguggguucgagccccacguugggcgcca

SEQ ID NO: 4 - RNA, 76 nt, Homo sapiens
gcccggauagcucagucgguagagcaucagacuuuuaaucugaggguccaggguucaagucccuguucgggcgcca